(19) United States
(12) Patent Application Publication
(10) Pub. No.: US 2004/0110176 A1
(43) Pub. Date: Jun. 10, 2004

(54) Fused protein having beta 1,2-n-acetylglucosaminyltransferase II activity and process for producing the same

(76) Inventors: Kazuhito Fujiyama, Osaka (JP); Tatsuji Seki, Osaka (JP); Shin-Ichiro Nishimura, Hokkaido (JP); Hiroaki Nakagawa, Hokkaido (JP); Susumu Nishiguchi, Shiga (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/469,145

(22) PCT Filed: Feb. 26, 2002
(86) PCT No.: PCT/JP02/01695

(30) Foreign Application Priority Data
Feb. 26, 2001 (JP) 2001-49955
Aug. 21, 2001 (JP) 2001-250165

(51) Int. Cl. C12Q 1/68; C07H 21/04; C08B 37/00; C12P 21/04; C12N 9/10

(52) U.S. Cl. 435/6; 435/69.7; 435/193; 435/320.1; 435/325; 536/23.2; 536/53

(57) ABSTRACT

To provide a fusion protein of a sugar-bonding protein and β1,2-N-acetylglucosaminyltransferase II, and a method for producing such proteins in E. coli cells.

### TECHNICAL FIELD

The present invention relates to a method for the inexpensive and efficient production of β1,2-N-acetyl glucosaminyltransferase II, which is a glycoprotein processing enzyme, and in particular to a fusion protein of a maltose-binding protein and β1,2-N-acetylglucosaminyltransferase II, as well as a method for producing this protein in E. coli cells. The present invention furthermore relates to modification of sugar chain structures attached to glycoprotein.

### BACKGROUND ART

Sugar chains comprising various monosaccharides linked by glycoside bonds exist in the form of intracellular organelle constituents, cell surface constituents, secretory proteins, and the like in cells. These sugar chain structures not only differ in ways specific to species and tissue, but also differ depending on development stage, disease, and the like even within the same species and tissue. It has thus become apparent that sugar chains function to provide proteins with physical properties conventionally attributed to proteins, such as thermal stability, hydrophilicity, electrical charge, and protease resistance, and that they also play a role in inter-cellular recognition such as that involved in development/differentiation, the nervous system, the immune system, and cancer metastasis, making them the subject of considerable recent scrutiny for potential application to various fields such as medicinal products.

These sugar chains are synthesized by glycosyltransferases within organisms. Glycosyltransferases are enzymes which transfer sugar from sugar nucleotides as sugar donors to acceptor sugar chains and extend the sugar chain. The specificity of sugar acceptor is highly stringent, it being commonly said that one glycoside bond is formed by one corresponding glycosyltransferase. These glycosyltransferases are important enzymes in terms of their use in glycobiological research, particularly the simple synthesis of useful sugar chains and the repair of natural sugar chains. However, the amounts of naturally occurring glycosyltransferase are limited, making it difficult to consistently provide large amounts for practical purposes.

β1,2-N-acetylglucosaminyltransferase II (GnTII) is an important enzyme involved in the formation of complex type sugar chains in glycoproteins, and has the action of transferring GlcNAc by means of β1-2 linkages to the acceptor Manα1-6(GlcNAcβ1-2Manα1-3)Manα1-4GlcNAcβ1-4GlcNAc-R using at least UDP-GlcNAc as the sugar donor, so as to produce GlcNAcβ1-2Manα1-6(GlcNAcβ1-2Manα1-3)Manβ1-4GlcNAcβ1-4GlcNAc-R (where R is asparagine residue, peptide, protein, or a low- or high-molecular weight compound which does not inhibit the activity of other glycosyltransferases). The consistent supply of such enzymes would be desirable because they are key enzymes in the formation of complex type sugar chains.

β1,2-N-acetylglucosaminyltransferase II has been purified from the organs of various species of animals. However, because organs are not readily available in large quantities, and because attempts at purification resulting in a single protein have proven to be extremely laborious, recombinant production is a promising alternative.

The cDNA coding for such enzymes has been isolated from various biological materials. For example, human β1,2-N-acetylglucosaminyltransferase II has been isolated by Tan et al. from leukocytes (Eur. J. Biochem., 231: 317-328 (1995)). It has also been isolated by R. Strasser et al. from Arabidopsis (Glycoconj. J., 16:787-791 (2000)).

The expression of proteins using microbes such as bacteria or yeast is beneficial in terms of cost for the mass production of recombinant proteins. However, as most proteins expressed in microbial expression systems occur in the form of insoluble inclusion bodies, and must be solubilized and renatured before purification, this cannot be considered an efficient option. In addition, even enzymes expressed in soluble form must undergo several purification processes, including various types of chromatographic treatment, in order to achieve a high degree of purification. Because this is considerably time-consuming and costly, it is not a satisfactory expression system for commercial industrial purposes.

An example of a method for expressing large amounts of the target protein and easily purifying the expression product is to express the target protein in the form of a fusion protein with glutathione-S-transferase (GST), protein A, or the like. In this method, fusion proteins containing GST can be readily purified by affinity column chromatography using glutathione as the ligand, and fusion proteins containing protein A can be readily purified by affinity column chromatography using IgG as the ligand. However, even in the case of above GST method the high possibility of producing inclusion body still remains. For these reasons, it is difficult to achieve a consistent supply of large quantities of β1,2-N-acetylglucosaminyltransferase II.

As noted above, most proteins in higher life forms occur in the form of glycoproteins, and their sugar chain structure is deeply involved in the recognition between cells and metabolic rates such as the absorption and degradation of substances in organisms. For that reason, when genes coding for physiologically active proteins derived from animals are expressed using yeast or plant hosts, for example, the sugar chain structure of the resulting glycoproteins differs from that of the original from the animal, often resulting in lower physiological activity (and sometimes no activity) or a higher degradation rate. It would be extremely useful if there was a method allowing such altered sugar chains to be modified back into the original sugar chain from the animal. Also, modifying sugar chains into ones that are different from the original bound sugar chains is expected to be of use in strengthening physiological functions or modifying physiological activity. Altering the expressing host or modifying the host through the introduction of a glycosyltransferase gene will alter the sugar chains binding to the protein targeted for expression but will not necessarily result in only the desired changes. A better method would be to modify the sugar chains of obtained glycoproteins in vitro. Several methods making use of transglycosylation by endoglycosidase have been proposed as such methods (such as Japanese Unexamined Patent Application (Kokai) 5-64594). However, endoglycosidase transglycosylation generally results in poor yields on sugar acceptors, and does not permit the efficient modification of sugar chains. Another method is to use exoglycosidase and a glycosyltransferase (Eur. J. Biochem., 191:75-73 (1990)), but the most that can be done is to modify the nonreducing terminal sugar residues, and this method thus cannot be considered to modify the entire sugar chain. There is also a method for using endoglycosidase and a glycosyltransferase (J. Am. Chem., Soc., 119:2114-2118 (1997)). In this method, hydrolysis is carried out with the endoglycosidase, and the sugar chains are then extended with the glycosyltransferase at the nonreducing terminal of the N-acetylglucosamine residues left on the protein, resulting in conversion to glycoproteins bound Sialyl-Lewis x tetrasaccharides thereto, but the bound sugar chains are the nonreducing terminal part of the glycoprotein sugar chains, making this method inadequate for modifying the entire sugar chain. One reason that current methods for modifying sugar chains with the use of exo- or endo-glycosidases and glycosyltransferases have not been adequate is that it has been difficult to ensure a stable supply of large amounts of β1,2-N-acetylglucosaminyltransferase II, the key enzyme for conversion.

An object of the invention is therefore to ensure the inexpensive and efficient supply of protein having β1,2-N-acetylglucosaminyltransferase II activity.

Another object of the invention is to modify sugar chain structures attached to glycoprotein.

### DISCLOSURE OF THE INVENTION

As a result of extensive research to overcome the drawbacks described above, the inventors discovered that when genetically engineered β1,2-N-acetylglucosaminyl transferase II is expressed in the form of a fusion protein with a sugar-binding protein such as a maltose-binding protein (MBP) in E. coli, β1,2-N-acetylglucosaminyltransferase II can be obtained in the form of a soluble protein, the protein can be readily purified by affinity chromatography using the specific affinity of sugar-binding proteins, and the resulting fusion protein has β1,2-N-acetylglucosaminyltransferase II activity (GnTII activity).

The inventors also discovered that GnTII can be obtained from the fusion protein using a protease that specifically cleaves the sequence at the site where the sugar-binding protein and GnTII are fused.

The inventors furthermore discovered that sugar chains on glycoprotein can be converted in vitro using GnTII capable of being consistently supplied in large amounts.

It was also discovered that such sugar chains on glycoprotein can be converted using immobilized enzymes. That is, the invention encompasses the following inventions.

1. A recombinant fusion protein of a sugar-binding protein and β1,2-N-acetylglucosaminyltransferase II (sugar-binding protein-GnTII).

2. A fusion protein according to item 1, wherein the β1,2-N-acetylglucosaminyltransferase II is derived from humans.

3. A fusion protein according to item 1, wherein the β1,2-N-acetylglucosaminyltransferase II is (a) a protein comprising the amino acid sequence in SEQ ID NO. 2, or (b) a protein with β1,2-N-acetylglucosaminyl transferase II activity, comprising the amino acid sequence in (a) above with one or more amino acids deleted, substituted, or added.

4. A protein according to item 3, wherein the β1,2-N-acetylglucosaminyltransferase II comprises at least the amino acid sequence 20-447 in the amino acid sequence in SEQ ID NO. 2.

5. A fusion protein according to items 1, wherein the β1,2-N-acetylglucosaminyl transferase II comprises an amino acid sequence in which amino acids corresponding to part or all of the protein transmembrane domain have been deleted.

6. A fusion protein according to item 1, comprising a protease recognition site between the sugar-binding protein and the β1,2-N-acetylglucosaminyl transferase II.

7. A fusion protein according to item 1, wherein the sugar-binding protein is a maltose-binding protein.

8. DNA coding for a fusion protein according to any of items 1 through 7.

9. An expression vector comprising DNA according to item 8.

10. A transformant resulting from transformation with an expression vector according to item 9.

11. A method for producing a sugar-binding protein/β1,2-N-acetylglucosaminyltransferase II fusion protein, comprising the following steps of:

(1) transforming E. coli using an expression vector to which DNA coding for a sugar-binding protein and DNA coding for β1,2-N-acetylglucosaminyltransferase II have been ligated in such a way that the two proteins are expressed in the form of a fusion protein under the control of a promoter capable of functioning in E. coli;

(2) cultivating the resulting transformants to produce a fusion protein of the sugar-binding protein and the β1,2-N-acetylglucosaminyltransferase II; and

(3) isolating the fusion protein from the resulting culture.

12. A method according to item 11, wherein the β1,2-N-acetylglucosaminyltransferase II is derived from humans.

13. A method according to item 12, wherein the DNA coding for the β1,2-N-acetylglucosaminyltransferase II comprises a nucleotide sequence coding for at least the amino acid sequence 29-447 in the amino acid sequence in SEQ ID NO. 2.

14. A method according to item 12, wherein the DNA coding for the β1,2-N-acetylglucosaminyltransferase II comprises at least the nucleotide sequence 85-1341 in the nucleotide sequence in SEQ ID NO. 1.

15. A method according to item 11, wherein the DNA coding for the β1,2-N-acetylglucosaminyltransferase II comprises a nucleotide sequence coding for an amino acid sequence in which amino acids corresponding to part or all of the protein transmembrane domain have been deleted.

16. A method according to item 11, wherein the DNA coding for the β1,2-N-acetylglucosaminyltransferase II comprises the nucleotide sequence in SEQ ID NO. 1 from which amino acids corresponding to part or all of the protein transmembrane domain have been deleted.

17. A method according to item 11, wherein the sugar-binding protein is a maltose-binding protein.

18. A method according to item 17, wherein the DNA coding for the maltose-binding protein is derived from pMAL-p2 or pMAL-c2.

19. A method according to item 11, wherein the fusion protein is isolated in the presence of divalent metal ions from the culture obtained in (3).

20. A method according to item 19, wherein the divalent metal is manganese (Mn²⁺).

21. A method for producing β1,2-N-acetyl glucosaminyltransferase II, comprising the step of isolating the β1,2-N-acetylglucosaminyltransferase II by eliminating the sugar-binding protein portion from the fusion protein obtained by a method according to any of items 11 through 20.

22. A method according to item 21, characterized in that the DNA coding for the sugar-binding protein comprises a nucleotide sequence coding for a protease recognition site on the C terminal end of the protein, and the sugar-binding protein portion is eliminated from the fusion protein through the action of a protease.

23. A method according to item 22, wherein the protease is blood coagulation factor Xa.

24. A method for converting sugar chains on glycoproteins to complex type sugar chains, comprising steps 1 through 4 below:

(step 1) allowing a glycosidase to act on glycoprotein sugar chains;

(step 2) allowing β1,2-N-acetylglucosaminyl transferase I to act, in the presence of UDP-GlcNAc, on the glycoproteins obtained in (step 1);

(step 3) allowing α-mannosidase to act on the glycoproteins obtained in (step 2); and

(step 4) allowing β1,2-N-acetylglucosaminyl transferase II to act, in the presence of UDP-GlcNAc, on the glycoproteins obtained in (step 3).

25. A method according to item 24, characterized in that at least one kind of glycosyltransferase is furthermore allowed to act after step 4.

26. A method according to item 25, wherein the glycosyltransferase is at least one selected from the group consisting of sialyltransferase, fucosyltransferase, galactosyltransferase, and N-acetylglucosaminyltransferase.

27. A method according to item 25, wherein at least one glycosyltransferase is an immobilized enzyme.

28. A method according to item 24, wherein the glycosidase is at least one selected from the group consisting of galactosidase, N-acetylglucosaminidase, fucosidase, sialidase, xylosidase, and mannosidase.

29. A method according to item 28, wherein the glycosidase is α-mannosidase.

30. A method according to item 29, wherein the glycosidase is α1,2-mannosidase.

31. A method according to item 24, wherein the α-mannosidase is α-mannosidase II.

32. A method according to item 24, wherein the β1,2-N-acetylglucosaminyltransferase II is a recombinant fusion protein according to any of items 1 through 7, or a β1,2-N-acetylglucosaminyltransferase II from which the sugar-binding protein has been eliminated by cleavage at the protease recognition site.

33. A method according to item 24, wherein at least one of the glycosidase, β1,2-N-acetyl glucosaminyltransferase I, α-mannosidase, or β1,2-N-acetylglucosaminyltransferase II is an immobilized enzyme.

34. A method according to item 24, wherein the glycoprotein is naturally derived.

35. A method according to item 24, wherein the glycoprotein is recombinant.

36. A method for converting sugar chains on glycoproteins to complex type sugar chains, comprising the following steps 1-3:

(step 1) allowing β1,2-N-acetylglucosaminyl transferase I to act, in the presence of UDP-GlcNAc, on glycoproteins having a structure wherein part or all of the sugar chain structures on the glycoproteins serve as the substrate for the β1,2-N-acetylglucosaminyl transferase I;

(step 2) allowing α-mannosidase to act on the glycoproteins obtained in (step 1); and

(step 3) allowing β1,2-N-acetylglucosaminyl transferase II to act, in the presence of UDP-GlcNAc, on the glycoproteins obtained in (step 2).

37. A method according to item 36, characterized in that at least one kind of glycosyltransferase is furthermore allowed to act after step 3.

38. A method according to item 37, wherein the glycosyltransferase is at least one selected from the group consisting of sialyltransferase, fucosyltransferase, galactosyltransferase, and N-acetylglucosaminyltransferase.

39. A method according to item 37, wherein at least one glycosyltransferase is an immobilized enzyme.

40. A method according to item 36, wherein the α-mannosidase is α-mannosidase II.

41. A method according to item 36, wherein the β1,2-N-acetylglucosaminyltransferase II is a recombinant fusion protein according to any of items 1 through 7, or a β1,2-N-acetylglucosaminyltransferase II from which the sugar-binding protein has been eliminated by cleavage at the protease recognition site.

42. A method according to any of items 36 through 41, wherein at least one of the β1,2-N-acetylglucosaminyl transferase I, α-mannosidase, or β1,2-N-acetylglucosaminyl transferase II is an immobilized enzyme.

43. A method according to item 36, wherein the glycoprotein is naturally derived.

44. A method according to item 36, wherein the glycoprotein is recombinant.

45. A method for converting sugar chains on glycoproteins to complex type sugar chains, comprising steps 1 through 3 below:

(step 1) allowing a glycosidase to act on glycoprotein sugar chains;

(step 2) allowing β1,2-N-acetylglucosaminyl transferase I to act, in the presence of UDP-GlcNAc, on the glycoproteins obtained in (step 1); and

(step 3) allowing β1,2-N-acetylglucosaminyl transferase II to act, in the presence of UDP-GlcNAc, on the glycoproteins obtained in (step 2).

46. A method according to item 45, characterized in that at least one kind of glycosyltransferase is furthermore allowed to act after step 3.

47. A method according to item 45, wherein the glycosyltransferase is at least one selected from the group consisting of sialyltransferase, fucosyltransferase, galactosyltransferase, and N-acetylglucosaminyltransferase.

48. A method according to item 46, wherein at least one glycosyltransferase is an immobilized enzyme.

49. A method according to item 45, wherein the glycosidase is at least one selected from the group consisting of galactosidase, N-acetylglucosaminidase, fucosidase, sialidase, xylosidase, and mannosidase.

50. A method according to item 45, wherein the β1,2-N-acetylglucosaminyltransferase II is a recombinant fusion protein according to any of items 1 through 7, or a β1,2-N-acetylglucosaminyltransferase II from which the sugar-binding protein has been eliminated by cleavage at the protease recognition site.

51. A method according to item 45, wherein at least one of the glycosidase, β1,2-N-acetylglucosaminyltransferase I, or β1,2-N-acetylglucosaminyltransferase II is an immobilized enzyme.

52. A method according to item 45, wherein the glycoprotein is naturally derived.

53. A method according to item 45, wherein the glycoprotein is recombinant.

54. A method for converting hybrid-type sugar chains on glycoproteins to complex type sugar chains, comprising steps 1 and 2 below:

(step 1) allowing a glycosidase to act on the sugar chains of hybrid-type glycoproteins; and

(step 2) allowing β1,2-N-acetylglucosaminyl transferase II to act, in the presence of UDP-GlcNAc, on the glycoproteins obtained in (step 1).

55. A method according to item 54, characterized in that at least one kind of glycosyltransferase is furthermore allowed to act after step 2.

56. A method according to item 55, wherein the glycosyltransferase is at least one selected from the group consisting of sialyltransferase, fucosyltransferase, galactosyltransferase, and N-acetylglucosaminyltransferase.

57. A method according to item 55, wherein at least one glycosyltransferase is an immobilized enzyme.

58. A method according to item 54, wherein the glycosidase is at least one selected from the group consisting of mannosidase, xylosidase, fucosidase, and β1,4-N-acetylglucosaminidase.

59. A method according to item 54, wherein the β1,2-N-acetylglucosaminyltransferase II is a recombinant fusion protein according to any of items 1 through 7, or a β1,2-N-acetylglucosaminyltransferase II from which the sugar-binding protein has been eliminated by cleavage at the protease recognition site.

60. A method according to item 54, wherein at least one of the glycosidase or β1,2-N-acetyl glucosaminyltransferase II is an immobilized enzyme.

61. A method according to item 54, wherein the glycoprotein is naturally derived.

62. A method according to item 54, wherein the glycoprotein is recombinant.

63. A method for converting high mannnose type sugar chains on glycoproteins into hybrid-type sugar chains, comprising the following steps 1 through 3, wherein at least one of the glycosidase, β1,2-N-acetylglucosaminyl transferase I, or β1,4-galactosyltransferase is an immobilized enzyme:

(step 1) allowing a glycosidase to act on high mannose-type sugar chains on glycoproteins;

(step 2) allowing β1,2-N-acetylglucosaminyl transferase I to act, in the presence of UDP-GlcNAc, on the glycoproteins obtained in (step 1); and

(step 3) allowing β1,4-galactosyltransferase to act, in the presence of UDP-Gal, on the glycoproteins obtained in (step 2).

64. A method according to item 63 for converting sugar chains on glycoproteins to hybrid-type sugar chains, characterized in that at least one kind of glycosyltransferase is furthermore allowed to act after step 3.

65. A method according to item 64, wherein the glycosyltransferase is at least one selected from the group consisting of sialyltransferase, fucosyltransferase, galactosyltransferase, xylosyltransferase, mannosyltransferase, and N-acetylglucosaminyltransferase.

66. A method according to item 64, wherein at least one glycosyltransferase is an immobilized enzyme.

67. A method according to item 63, wherein the glycosidase is at least one selected from the group consisting of galactosidase and α-mannosidase.

68. A method according to item 63, wherein the α-mannosidase is α1,2-mannosidase.

69. A method according to item 63, wherein the glycoprotein is naturally derived.

70. A method according to any of times 63, wherein the glycoprotein is recombinant.

### BRIEF DESCRIPTION OF THE DRAWINGS

FIG. 1 is an outline of the preparation of a vector in the invention (Example 2);

FIG. 2 shows the results of HPLC analysis indicating the reactivity of the fusion protein solution (MBP-hGnTII) obtained in Example 3. FIG. 2A gives the results of analysis for the standard pyridylaminated oligosaccharides (Takara Shuzo) represented by Formulas 2 and 3, and FIG. 2B gives the results from 3 hours of reaction using the pyridylaminated oligosaccharide represented by Formula 2 as the substrate (Example 4);

FIG. 3 shows the optimal reaction temperature for the MBP-hGnTII obtained in Example 3 (Example 6). The activity at 40° C. was considered 100%;

FIG. 4 shows the thermal stability of the MBP-hGnTII obtained in Example 3 (Example 7). The activity at 10° C. was considered 100%;

FIG. 5 shows the optimal pH for the MBP-hGnTII obtained in Example 3 (Example 8). The activity in cacodylate buffer (pH 7.0) was considered 100%;

FIG. 6 shows the pH stability of the MBP-hGnTII obtained in Example 3 (Example 9). The activity in glycine buffer (pH 8.8) was considered 100%;

FIG. 7 shows the sugar chain analysis of samples obtained in the steps in Example 10, as determined by normal phase HPLC;

FIG. 8 shows the results of mass spectrum analysis of the main peak in step 4 in Example 10;

FIG. 9 illustrates an embodiment of the invention as described on items 24 through 35;

FIG. 10 illustrates an embodiment of the invention as described on items 36 through 44;

FIG. 11 illustrates an embodiment of the invention as described on items 45 through 53;

FIG. 12 illustrates an embodiment of the invention as described on items 54 through 62; and

FIG. 13 illustrates an embodiment of the invention as described on items 63 through 70.

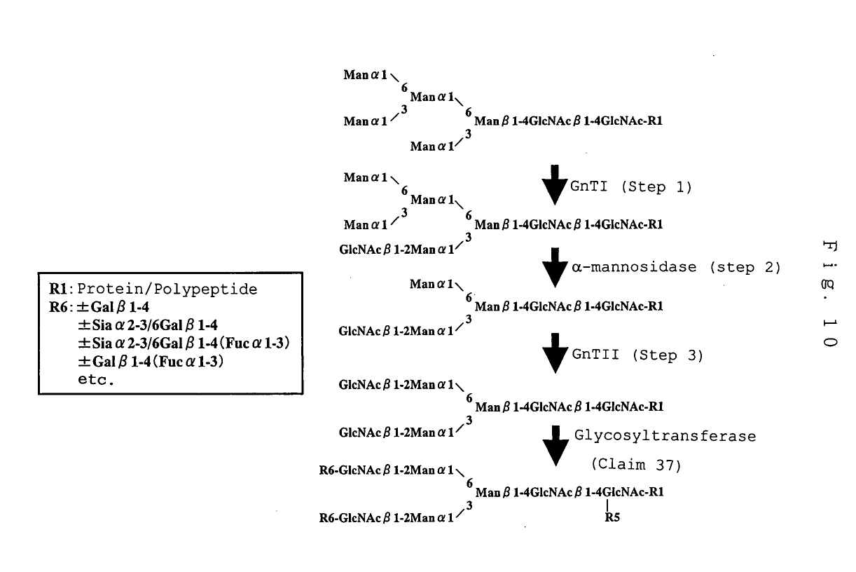

In FIGS. 9 through 13, the symbols l, m, n, and x represent integers from 0 to 20.

### DETAILED DESCRIPTION OF THE INVENTION

The present invention is characterized in that proteins with GnTII activity are reconstructed in the form of fusion proteins of GnTII and a sugar-binding protein, the fusion protein being a soluble protein.

The present invention is also characterized by the fact that GnTII can be expressed as a fusion protein with a sugar-binding protein in E. coli cells to produce GnTII in the form of a soluble protein, and the fact that the specific affinity of the sugar-binding protein can be exploited to allow the fusion protein and GnTII to be readily purified in large amounts.

The present invention is furthermore characterized in that sugar chains on glycoprotein can be converted using a glycosidase and glycosyltransferase.

Fusion Protein of the Invention

In the present invention, the fusion protein of a sugar-binding protein such as MBP and GnTII (sometimes abbreviated as MBP-GnTII below for fusion proteins containing maltose-binding protein) may be in any form, as long as it retains the specific affinity of the sugar-binding protein, that is, the high affinity for maltose or oligosaccharides and polysaccharides containing maltose segment, as well as the enzyme activity of GnTII, that is, the activity in transferring GlcNAc from UDP-GlcNAc as the sugar donor via β1-2 linkages to the acceptor Manα1-6(GlcNAcβ1-2Manα1-3)Manβ1-4GlcNAcβ1-4GlcNAc-R (where R is asparagine residue, peptide, protein, or low- or high-molecular weight compound which does not inhibit the activity of other glycosyltransferases), so as to produce GlcNAcβ1-2Manα1-6(GlcNAcβ1-2Manα1-3)Manβ1-4GlcNAcβ1-4GlcNAc-R. Furthermore, as indicated in Formula 1, GnTII obtained after the elimination of the sugar-binding protein portion has at least inherent enzyme activity, that is, activity in transferring GlcNAc from UDP-GlcNAc as the sugar donor via β1-2 linkages to the acceptor Manα1-6(GlcNAcβ1-2Manα1-3)Manβ1-4GlcNAcβ1-4GlcNAc-R, so as to produce GlcNAcβ1-2Manα1-6(GlcNAcβ1-2Manα1-3)Manβ1-4GlcNAcβ1-4GlcNAc-R.

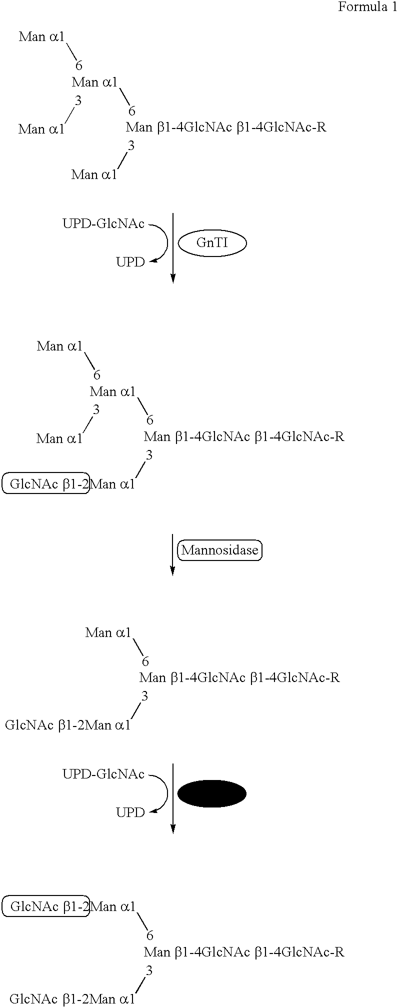

The GnTII will preferably be linked to the C terminal of the sugar-binding protein in the fusion protein. Even more preferably, the fusion protein will have a structure allowing the sugar-binding protein and GnTII to be readily cleaved by enzymatic or chemical means at the fusion site.

Sugar-binding proteins are proteins having high affinity for monosaccharides as well as oligosaccharides and polysaccharides, examples of which include various lectins, maltose-binding protein, cellulose-binding protein, and chitin-binding protein. Maltose-binding protein, cellulose-binding protein, chitin-binding protein, and the like are preferred, and maltose-binding protein (MBP) is especially desirable.

The sugar-binding proteins can be derived from any species, but are preferably from prokaryotes such as bacterial cells, and even more preferably from E. coli cells.

MBP need not necessarily have the complete sequence, and may include part of the sequence, provided that they have sites with affinity specific to maltose or oligosaccharides with maltose segment (such as maltotriose) or polysaccharides with maltose segment (such as amylose). Examples include proteins which have the known amino acid sequence with one or more amino acids deleted, substituted, or added, and has a domain with affinity to maltose or oligosaccharides with maltose segment (such as maltotriose) or polysaccharides with maltose segment (such as amylose).

DNA derived from pMAL-p2 or pMAL-c2 (both from New England Biolabs) were used as the genes DNA coding for MBP in the present invention.

The sugar-binding proteins of the present invention should especially include an amino acid sequence which is recognized by a protease with high substrate specificity at the C terminal of the protein. Examples of such proteases include factor Xa, thrombin, and renin. The protease recognition sequence is preferably the factor Xa recognition sequence, that is, Ile-Glu-gly-Arg (SEQ ID NO. 3). Such a protease recognition sequence can be introduced to the C terminal of the sugar-binding protein so as to allow the action of the protease after purification of the MBP-GnTII to readily eliminate the MBP.

In a preferred embodiment, the fusion protein will include a spacer sequence consisting of about 5 to 15 amino acid residues between the sugar-binding protein moiety and the protease recognition site. The spacer is intended to put some distance between the sugar-binding protein and the GnTII so as to deduce the possibility of intramolecular interaction in the fusion protein, and to enhance the affinity for maltose or amylose in the MBP portion of MBP-GnTII fusion proteins. pMAL-p2 or pMAL-c2 (by New England Biolabs) including the spacer sequence in SEQ ID NO. 9 can be used in the present invention.

GnTII which is derived from any species is applicable to the present invention but is preferably from mammals and more preferably from humans. Furthermore, in the present invention, the GnTII need not contain the full length GnTII sequence, and may include part of the sequence, provided that the GnTII activity is preserved. For example, it may be (a) a protein comprising the amino acid sequence in SEQ ID NO. 2 or (b) a protein that comprises the amino acid sequence in (a) with one or more amino acids deleted, substituted, or added, and that has β1,2-N-acetylglucosaminyltransferase II activity.

Because GnTII is a membrane protein in the Golgi apparatus, a particularly desirable embodiment of the invention features the use of GnTII lacking part or all of its highly hydrophobic transmembrane domain, in the interests of allowing the MBP-GnTII to be produced in the form of a soluble protein. Specifically, the invention should include the amino acid sequence from at least 29-447 in the amino acid sequence in SEQ ID NO. 2.

It may also be a protein that comprises the amino acid sequence from at least 29-447 in the amino acid sequence of SEQ ID NO. 2, with one or more amino acids deleted, substituted, or added, and that has β1,2-N-acetylglucosaminyltransferase II activity.

Preparation of the Fusion Protein of the Invention

In the present invention, the sugar-binding protein-GnTII fusion protein can be obtained by cultivating, in a suitable medium, the transformant, which has been obtained upon the transformation of E. coli with an expression vector comprising chimera DNA ligated in such a way—namely, in-frame—that the DNA coding for the sugar-binding protein and the DNA coding for the GnTII are transcribed and translated in the form of a fusion protein having the properties described above.

In the present invention, the DNA coding for GnTII may be derived from any species, but is preferably from mammals, and even more preferably from humans. The DNA coding for the GnTII in the present invention need not include the entire coding region of the GnTII, and may include a part of the coding region, provided that the transcription/translation product retains GnTII activity. Because GnTII is a membrane protein in the Golgi apparatus, a particularly desirable embodiment of the invention features the use of GnTII lacking the region coding for the highly hydrophobic transmembrane domain, in the interests of allowing the sugar-binding protein-GnTII to be produced in the form of a soluble protein.

Based on the known nucleotide sequence coding for GnTII (such as Eur. J. Biochem., 231:317-328 (1995)) or the like, the DNA coding for the GnTII can be prepared by any known method. For example, a pair of suitable oligonucleotide primers covering part or all of the GnTII coding region can be synthesized based on the known GnTII nucleotide sequence, and the total RNA or poly A⁽⁺⁾RNA or chromosomal DNA extracted from cells or tissue expressing GnTII can be used as template in RT-PCR or PCR to clone the DNA. A suitable restriction enzyme recognition sequence can be added to the terminal of the oligonucleotide primers that are used, in order to facilitate subsequent cloning to the vector.

Alternatively, a suitable oligonucleotide probe can be synthesized based on the known GnTII nucleotide sequence, a cDNA library can be prepared in the usual manner from cells or tissue expressing the GnTII, and the DNA coding for GnTII can be obtained from the library by plaque (or colony) hybridization.

Furthermore, antibody is prepared in the usual manner by using partially or completely purified GnTII as antigen, the DNA coding for the GnTII can be cloned from cDNA library prepared in the usual manner from cells or tissue expressing the GnTII by using the antibody.

Alternatively, DNA can be synthesized based on the known GnTII nucleotide sequence using a DNA/RNA automatic synthesizer in such a way that part of the sense strand partial sequence overlaps part of the antisense strand partial sequence, and the longer partial sequences are repeatedly obtained in the form of double-stranded DNA by PCR, giving the desired DNA sequence.

In a preferred embodiment of the invention, the DNA is comprised of part or all of the DNA coding for human β1,2-N-acetylglucosaminyltransferase II (hGnTII). Examples include DNA comprising a nucleotide sequence coding for the amino acid sequence at least 29-447 in the amino acid sequence in SEQ ID NO. 2, and preferably the nucleotide sequence at least 85-1341 in the nucleotide sequence in SEQ ID NO. 1. Alternatively, the DNA coding for hGnTII may be DNA comprising a nucleotide sequence coding for an amino acid sequence in which an amino acid sequence corresponding to part or all of the transmembrane domain of the protein has been deleted, and preferably comprises a nucleotide sequence in which a nucleotide sequence coding for an amino acid sequence corresponding to part or all of the transmembrane domain of the protein has been deleted. Based on hydrophobic plot results, the transmembrane domain of hGnTII is assumed to be the portion represented by amino acid sequence 10-28 in the amino acid sequence of SEQ ID NO. 2. The DNA coding for the domain is therefore preferably the nucleotide sequence 28-84 in the nucleotide sequence in SEQ ID NO. 1.

Of course, the DNA coding for the hGnTII may be DNA comprising a nucleotide sequence coding for all of the amino acid sequence in SEQ ID NO. 2, and is preferably DNA comprising all of the nucleotide sequence in SEQ ID NO. 1.

Examples of DNA coding for the sugar-binding protein in the present invention include DNA coding for the various proteins described above, and preferably DNA coding for maltose-binding protein, cellulose-binding protein, or chitin-binding protein, especially DNA coding for maltose-binding protein.

The DNA may be from any species, but is preferably from prokaryotes such as bacterial cells, especially E. coli cells.

The DNA coding for the MBP need not necessarily include the entire coding region, and may include part of the coding region, provided that it codes for a translation product having affinity for maltose, oligosaccharides with maltose segment (such as maltotriose), or polysaccharides with maltose segment (such as amylose). Similarly, the sugar-binding protein need not necessarily include the entire coding region, and may include part of the coding region, provided that it codes for a translation product with affinity for the binding sugar.

MBP derived from E. coli is secretory protein localized in periplasm, and the initial translation product includes a signal peptide at the N terminal. DNA coding for MBP in the present invention may or may not include a nucleotide sequence coding for a signal peptide (signal codon) capable of functioning in E. coli hosts. Because E. coli is a gram negative bacterium with an outer membrane outside of the cell wall, even when the DNA coding for MBP includes a signal codon, there is little possibility of the expressed MBP-GnTII being secreted in media. However, if the MBP-GnTII accumulates in the periplasmic space, spheroplasts can be formed without completely rupturing the cells, thereby allowing the fusion protein to be recovered, and thus making subsequent purification easier to manage. In such cases, however, the transmembrane domain of GnTII should be eliminated. When the transmembrane domain is present, the MBP-GnTII might remain in the inner membrane without reaching the periplasmic space, thus complicating the recovery of the fusion protein.

The DNA coding for MBP in the present invention should especially include a nucleotide sequence coding for an amino acid sequence that is cleaved upon being recognized by a protease with high substrate specificity at the C terminal of the protein (protease recognition site). Examples of such proteases include factor Xa, thrombin, and renin. The protease recognition sequence is preferably an factor Xa recognition sequence, that is, Ile-Glu-gly-Art (SEQ ID NO. 3). Such a protease recognition sequence can be introduced to the C terminal of the sugar-binding protein so as to allow the action of the protease after purification of the MBP-GnTII to readily eliminate the MBP.

In a preferred embodiment, the DNA coding for the fusion protein will include a nucleotide sequence coding for a spacer of about 5 to 15 amino acid residues between the sugar-binding protein coding region and the protease recognition site, or between the sugar-binding protein coding region and the GnTII coding region. The spacer is intended to put some distance between the sugar-binding protein and the GnTII so as to minimize the possibility of intramolecular interaction in the fusion protein, and to enhance the affinity for maltose or amylose in the MBP portion of MBP-GnTII fusion proteins.

Based on the well known nucleotide sequence coding foe MBP (such as E. coli MBP (Duplay P. et al., J. Biol. Chem., 259:10606-10613 (1984)), the DNA coding for MBP can be cloned by the same well known means as examples for the DNA coding for GnTII, and the protease recognition sequence and/or spacer sequence can be introduced to the C terminal of the MBP in the usual manner. A preferred embodiment of the invention will include the spacer sequence in SEQ ID NO. 8.

DNA coding for an E. coli-derived MBP can be obtained from commercially available vectors such as pMAL-p2 (including a signal codon) or pMAL-c2 (no signal codon) by New England Biolab.

A suitable restriction enzyme recognition site can be attached in the usual manner to the terminal of the DNA coding for sugar-binding proteins in order to facilitate construction of the chimera DNA coding for the sugar-binding protein-GnTII fusion protein. In cases where a restriction enzyme recognition site is added to the N terminal of GnTII when DNA coding for GnTII is cloned in a vector, the same restriction enzyme recognition site (or another restriction enzyme recognition site producing the same sticky end) should be incorporated in the DNA coding for the sugar-binding protein.

The expression vector of the present invention may be any expression vector in which the chimera DNA coding for the sugar-binding protein-GnTII fusion protein is under the control of a promoter capable of functioning in E. coli. The promoter region includes a −35 region and −10 region which are consensus sequences determining the binding site of the RNA polymerase. An induction enzyme promoter region should be used as the system for expressing large amounts of the desired recombinant protein. Examples of such promoter regions include trp promoter, lac promoter, recA promoter, lpp promoter, and tac promoter. These promoter regions also include operators to which repressor proteins bind. The addition of an inducer (such as lactose or IPTG when using a lac promoter) will inhibit the repressor protein binding to the operator, resulting in the expression of large amounts of the recombinant protein under the control of the promoter. The expression vector also contains a consensus Shine-Dalgarno (SD) sequence upstream of the translation start codon. The expression vector also includes a transcription termination signal, that is, a terminator region, down stream of the chimera DNA coding for the sugar-binding protein-GntII fusion protein. Normally used natural or synthetic terminators can be employed as the terminator region. The expression vector of the invention must include an origin of replication capable of autonomous replication in E. coli hosts, in addition to the above promoter region and terminator region. Examples of such origins of replication include ColE1ori and M13ori.

The expression vector of the present invention should also include a selection marker for selection of transformants. Resistance gene against various antibiotics such as tetracycline, ampicillin, and kanamycin can be used as the selection marker. When the E. coli host is an auxotrophic mutant, wild type genes complementing the auxotrophic properties can be used as the selection marker.

The transformants of the invention can be prepared by transforming the E. coli host with the expression vector of the invention. The host cell line is not particularly limited. Examples include commercially available strain such as XL1-Blue, BL-21, JM107, TB1, JM109, C600, DH5α, HB101, and so on.

The expression vector can be introduced into the host cells using a conventionally known method. Examples include the method of Cohen et al (calcium chloride method) (Proc. Natl. Acad. Sci. USA, 69:2110 (1972)), the protoplast method (Mol. Gen. Genet., 168:111 (1979)), the competent cell method (J. Mol. Biol., 56:209 (1971)), and electroporation.

The sugar-binding protein-GnTII fusion protein can be obtained by recovering sugar-binding protein-GnTII fusion protein from culture that has been obtained upon cultivation, in suitable media, transformants expressing the expression vector which includes the chimera DNA coding for the sugar-binding protein-GnTII fusion protein.

The media should contain carbohydrates such as glucose, fructose, glycerol, or starch as carbon sources. Inorganic or organic nitrogen sources should also be included (such as ammonium sulfate, ammonium chloride, casein hydrolysates, yeast extracts, polypeptone, bactotryptone, and beef extract). Such carbon and nitrogen sources need not be used in pure form. Those of low purity are advantageous because they contain an abundance of inorganic nutrients or trace amounts of growth factor. Other nutrient sources (such as inorganic salts (for example, sodium diphosphate or potassium diphosphate, dibasic potassium phosphate, magnesium chloride, magnesium sulfate, and calcium chloride), vitamins (such as Vitamin B1), antibiotics (such as ampicillin and kanamycin)) may also be added as desired to the medium.

The transformants are usually cultivated for 1 to 150 hours at a temperature of 18 to 40° C., and preferably 20 to 35° C., at a pH of 5.5 to 8.5, and preferably 6 to 8, but these can be modified as needed depending on culture conditions and the scale of the culture.

To avoid slowing down the growth rate during the production process of the target protein when the cultivation is managed in large tanks, the medium inoculated with a small amount of cells should be cultivated for 1 to 24 hours, and the resulting culture should then be inoculated into the large tanks.

When the expression of the sugar-binding protein-GnTII fusion protein is controlled by an induction protein gene promoter system, the inducer may be added after the start of cultivation, but is preferably added during the initial logarithmic growth phase. Bacterial cell growth can be monitored by measuring the optical density of the culture broth at 660 nm. When, for example, a lac promoter or tac promoter is used, isopropylthio-β-D-galactoside (IPTG) can be added as the inducer in a concentration of between 0.1 and 1.0 mM, for example, when the optical density reaches 0.4 to 0.6 at 660 nm. The period in which the inducer is added and the rate at which it is added can be modified as needed depending on the culture conditions, the scale of the culture, the type of inducer, and the like.

In the method of the invention, the sugar-binding protein-GnTII fusion protein can be purified in a single step by treating fractions containing the fusion protein by affinity chromatography using an insoluble carrier bound various sugar residues as ligand specifically binding to the sugar-binding protein.

For example, when the sugar-binding protein is MBP and the DNA coding for the MBP lacks signal peptide region, the MBP-GnTII fusion protein is localized in the cytoplasm. In such cases, therefore, after cultivation the cultured cells can be recovered by filtration or centrifugation, the cells can be ruptured by lysozyme and surfactant treatment, ultrasonication, or the like, and the resulting cell-free extract can apply to affinity chromatography.

When the DNA coding for MBP which have a signal peptide region, on the other hand, it is very likely that the expressed MBP-GnTII fusion protein will be secreted and will accumulate in the periplasmic space. As such, in these cases, the MBP-GnTII fusion protein is extracted from spheroplasts prepared from cultivated cell by lysozyme treatment or the like, the spheroplasts can be removed by filtration or centrifugation, and the resulting supernatant can then apply to affinity chromatography.

In the present invention, fractions containing sugar-binding protein-GnTII fusion protein should be purified as described above in the presence of divalent metal ions. Specifically, the cells are harvested from the culture in the presence of divalent metal ions after cultivation, and the cells are ruptured or the like to obtain fractions containing sugar-binding protein-GnTII fusion protein.

Examples of divalent metal ions include manganese ions (Mn²⁺) and magnesium ions (Mg²⁺), but manganese ions are preferred. Manganese ions can be used in the form of manganese salts such as manganese chloride, manganese sulfate, manganese nitrate, or manganese bromide.

Amylose resin comprising amylose immobilized on agarose beads (such as amylose resin columns by New England Biolabs) can be used as the adsorbent for affinity chromatography in the case of MBP, for example, but other ligands for MBP and other insoluble matrices (such as cellulose, dextran, and synthetic polymers) may also be used.

The adsorbent is added to the fractions containing the sugar-binding protein-GnTII fusion protein prepared according to the above and the mixture is agitated for a suitable period of time. The mixture is then filtered to separate the adsorbent, and the adsorbent is washed. A suitable concentration of eluate containing a sugar (such as maltose in the case of MBP) to inhibit binding between the adsorbent and the sugar-binding protein is furthermore added and mixed for a suitable period of time, and the mixture is then filtered, so that the purified sugar-binding protein-GnTII is obtained in the filtrate. Alternatively, it can be obtained by packing a column with adsorbent, applying fractions containing sugar-binding protein-GnTII fusion protein onto the column, washing the column with a suitable buffer, and then applying a suitable concentration of eluate to elute the sugar-binding protein-GnTII adsorbed to the column.

MBP-GnTII fusion protein of the present invention thus obtained has GnTII activity and can be used as an enzyme.

Preparation of GnTII

As described above, in a preferred embodiment of the invention, the sugar-binding protein-GnTII fusion protein contains an amino acid sequence that is cleaved by a sequence-specific protease at the fused site, thus allowing the protease to act on the sugar-binding protein-GnTII purified in the manner described above, so that the sugar-binding protein portion is eliminated, giving the desired GnTII. The protease is preferably factor Xa. pMAL-p2 or pMAL-c2 (New England Biolabs) containing the factor Xa recognition sequence of SEQ ID NO. 3 can be used in the present invention. The reaction temperature, solution pH, reaction time, and the like used when the protease is allowed to act on the sugar-binding protein-GnTII fusion protein can be adjusted as desired according to the type of protease being used, but in the case of factor Xa, the reaction can be carried out for 1 to 25 hours at 4 to 40° C. in neutral buffer to cleave the sugar-binding protein and the GnTII. After the completion of the reaction, the above adsorbent is added to the reaction solution and mixed for a suitable period of time to allow just the free sugar-binding protein to be adsorbed to the adsorbent, and the adsorbent can thus be filtered to purify the GnTII alone. Alternatively, the GnTII can be purified by applying the reaction solution onto a column packed with the adsorbent and collecting the fractions that pass through the column.

Conversion of Glycoprotein Sugar Chains

As used in the present Specification, high mannose type sugar chains refer to sugar chains with a structure in which only mannose is linked to the mannose residues at the non-reducing terminal of Manα1-6(Manα1-3)Manβ1-4GlcNAcβ1-4GlcNAc-, which is the core structure of N-linked sugar chains. Specific examples include sugar chains with a Manα1-2Manα1-6(Manα1-2Manα1-3)Manα1-6(Manα1-2Manα1-2Manα1-3)Manβ1-4GlcNAcβ1-4GlcNAc- structure, but also include those in which one or more mannose residues are degraded during the intracellular maturation of the sugar chain structure. Structures including α1-3-linked mannose or α1-6-linked mannose and galactose are also known (Clinton E. Ballou et, al., Proc. Natl. Acad. Sci. USA 91:9327 (1994)).

As used in the present Specification, complex type sugar chains refer to sugar chains with a structure in which the basic skeleton is a structure in which N-acetylglucosamine is the only sugar linked to the mannose residues at the non-reducing terminal of Manα1-6(Manα1-3)Manβ1-4GlcNAcβ1-4GlcNAc-, which is the core structure of N-linked sugar chains. Galactose, sialic acid, fucose, xylose, N-acetylglucosamine, and the like may also be linked to the basic skeleton. Specific examples include sugar chains with structures such as GlcNAcβ1-2Manα1-6(GlcNAcβ1-2Manα1-3)Manβ1-4GlcNAcβ1-4GlcNAc-, or Galβ1-4GlcNAcβ1-2Manα1-6(Galβ1-4GlcNAcβ1-2Manα1-3)Manβ1-4GlcNAcβ1-4GlcNAc-, or Siaα2-6Galβ1-4GlcNAcβ1-2Manα1-6(Siaα2-6Galβ1-4GlcNAcβ1-2Manα1-3)Manβ1-4GlcNAcβ1-4GlcNAc-. The N-acetylglucosamine need not necessarily be linked by the β1-2 linkage to the non-reducing end of the core structure Manα1-6(Manα1-3)Manβ1-4GlcNAcβ1-4GlcNAc- of the N-linked sugar chain, but may also be linked, for example, by β1-3, β1-4, β1-6, α1-2, α1-3, α1-4, and α1-6 linkages.

As used in the present Specification, hybrid-type sugar chains refer to sugar chains with a structure in which the basic skeleton is a structure in which mannose is the sugar linked to the α1-6 mannose residues at the non-reducing terminal of Manα1-6(Manα1-3)Manβ1-4GlcNAcβ1-4GlcNAc-, which is the core structure of N-linked sugar chains, and N-acetylglucosamine is the sugar linked to the α1-3 mannose residues. Galactose, sialic acid, fucose, xylose, N-acetylglucosamine, and the like may also be linked to the basic skeleton. Specific examples include sugar chains with structures such as Manα1-6(Manα1-3)Manα1-6(Siaα2-3Galβ-4GlcNAcβ1-2Manα1-3)(Xylβ1-2)Manβ1-4GlcNAcβ1-4(Fucα1-3)GlcNAc.

Examples of glycoproteins which have been expressed by genetic engineering and which have high mannose type sugar chains with 2 to 9 mannose residues include glycoproteins obtained by genetic engineering using hosts such as CHO or other mammal cells, yeasts, insect cells, molds, chicken cells or eggs, algae, plant cells or other eukaryote cells, or mammal, insect, or plant hosts.

Hosts for the production of recombinant proteins with modified sugar chain structures have been improved using recent genetic engineering techniques. It is reported that the recombinant yeast cell comprising the Aspergillus saitoi α1,2-mannosidase gene incorporated into triple mutant (och1, mnn1, mnn4) Saccharomyces cerevisiae produce glycoprotein bound high mannose type sugar chain containing 5 mannose residues, in which the glycoprotein sugar chain structure is an optimal substrate for β1,2-N-acetylglucosaminyltransferase I (Chiba Y., et al., J. Biol. Chem., 273:26298-26304 (1988)). The introduction and expression of a useful exogenous protein gene in this recombinant yeast host results in the recombinant glycoprotein bound high mannose type sugar chain structure with five mannose residues, and the action of β1,2-N-acetylglucosaminyl transferase I can result in the conversion to a glycoprotein having a GlcNAcβ1-2Manα1-3Manβ1-4 moiety. Alternatively, a useful exogenous gene can be introduced into triple mutant (och1, mnn1, mnn4) Saccharomyces cerevisiae yeast and expressed. The subsequent action of Aspergillus saitoi α1,2-mannosidase or mannosidase I and followed by the action of β1,2-N-acetylglucosaminyl transferase I can result in the conversion to a glycoprotein with a GlcNAcβ1-2Manα1-3Manβ1-4 moiety.

Glycoproteins with a sugar chain structure consisting of 5 to 8 mannose residues have been obtained with another triple mutant (och1, mnn1, alg3) Saccharomyces cerevisiae yeast (Nakanishi-Shindo Y., J. Biol. Chem., 268:26338-26345 (1993)). The action of Aspergillus saitoi α1,2-mannosidase or mannosidase I, followed by the action of β1,2-N-acetylglucosaminyltransferase I, can result in the conversion to a glycoprotein with a GlcNAcβ1-2Manα1-3Manβ1-4 moiety.

Arabidopsis thaliana plant cells with a mutation in the β1,2-N-acetylglucosaminyltransferase I gene have been discovered (von Schaewen A. et al., Plant Physiol., 102:1109-1113(1993)). This mutant plant gives a glycoprotein with a high mannose type sugar chain containing 5 mannose residues. Manα1-6(Manα1-3)Manα1-6(Manα1-3)Manβ1-4 with a sugar chain structure containing of 5 mannose residues is a more desirable substrate for β1,2-N-acetyl glucosaminyltransferase I. Application of the cDNA of β1,2-N-acetyl glucosaminyltransferase I cloned from plants to tobacco plants for antisense method or cosupression method gave plants with reduced β1,2-N-acetylglucosaminyltransferase activity (Wenderoth I. and von Schaewen A., Plant Physiol., 123:1097-1108(2000)). The incorporation and expression of a useful exogenous gene in such recombinant plant hosts can result in a high mannose type sugar chain structure containing 5 mannose residues, and the in vitro action of β1,2-N-acetylglucosaminyltransferase I can result in the conversion to glycoproteins with a GlcNAcβ1-2Manα1-3Manβ1-4 moiety.

Regardless of the type of protein, all glycoproteins are encompassed, such as RNaseB.

In step 1 of item 24 or step 1 of item 63, the sugar chain structure can be converted from, for example, a hybrid-type sugar chain (items 24 and 63) or a high mannose type sugar chain (item 24) (see FIGS. 9 and 13) to the Manα1-6 (Manα1-3) Manα1-6 (Manα1-3) Manβ1-4GlcNAcβ1-4GlcNAc structure, and α1,2-mannosidase is the preferred glycosidase to use in these steps. α1,2-mannosidase is an enzyme that hydrolyzes mannose residues linked by the α1,2 linkage. Mannosidase I and Man₉-mannosidase are also included in α1,2-mannosidase in the present invention. When the outer chain of sugar chain on the glycoprotein intended to convert its sugar chain includes α1-3-linked mannose or α1-6-linked mannose, α-mannosidase can also be used. Furthermore, at least one selected from the group consisting of xylosidase, fucosidase, N-acetylglucosaminidase, galactosidase, sialidase, and the like can also be used as the glycosidase when xylose, fucose, N-acetylglucosamine, galactose, sialic acid, or the like is included in sugar chains on the glycoprotein intended to convert its sugar chain.

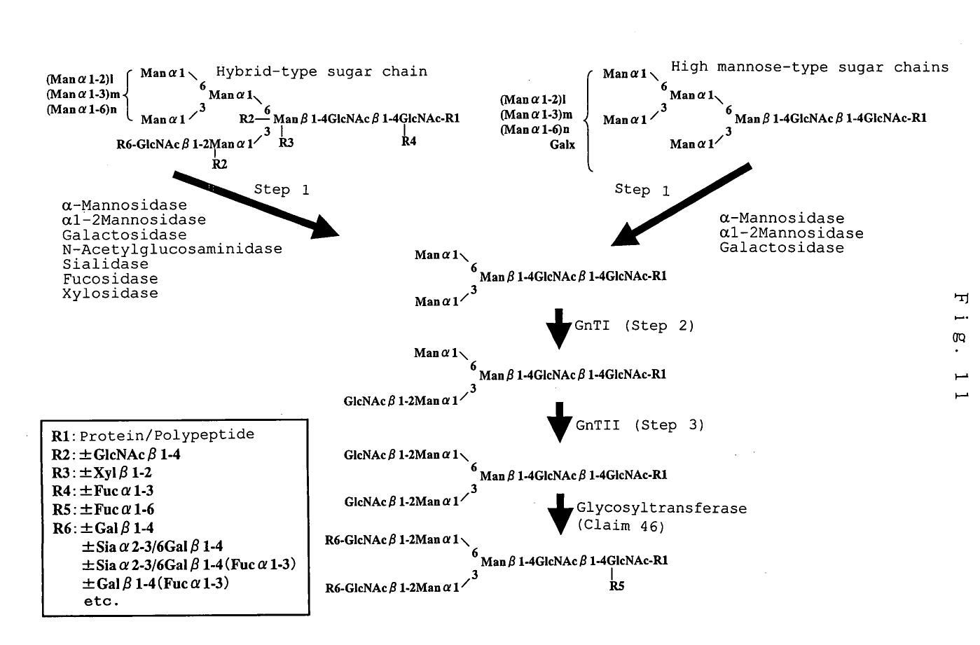

In step 3 of item 24 or step 2 of item 36, the sugar chain structure can be converted from, for example, a Manα1-6 (Manα1-3) Manα1-6 (GlcNAcβ1-2Manα1-3) Manβ1-4GlcNAcβ1-4GlcNAc structure to a Manα1-6(GlcNAcβ1-2Manα1-3)Manβ1-4GlcNAcβ1-4GlcNAc structure (see FIGS. 9 and 10), and the α-mannosidase used in this case is an enzyme that hydrolyzes mannose linked by the α1,2-linkage, α1,3-linkage, α1,4-linkage, or α1,6-linkage. α-mannosidase II is an enzyme that hydrolyzes mannose linked by the α1,3-linkage or α1,6-linkage, but its substrate specificity is more stringent, making it a more desirable enzyme for this process in terms of catalyzing the reaction Manα1-6(Manα1-3)Manα1-6(GlcNAcβ1-2Manα1-3)Manβ1-4GlcNAcβ1-4GlcNAc→Manα1-6(GlcNAcβ1-2Manα1-3)Manβ1-4GlcNAcβ1-4GlcNAc, without deleting the mannose up to GlcNAcβ1-2Manα1-3)Manβ1-4GlcNAcβ1-4GlcNAc (J. Biol. Chem. 266: 16876 (1991)).

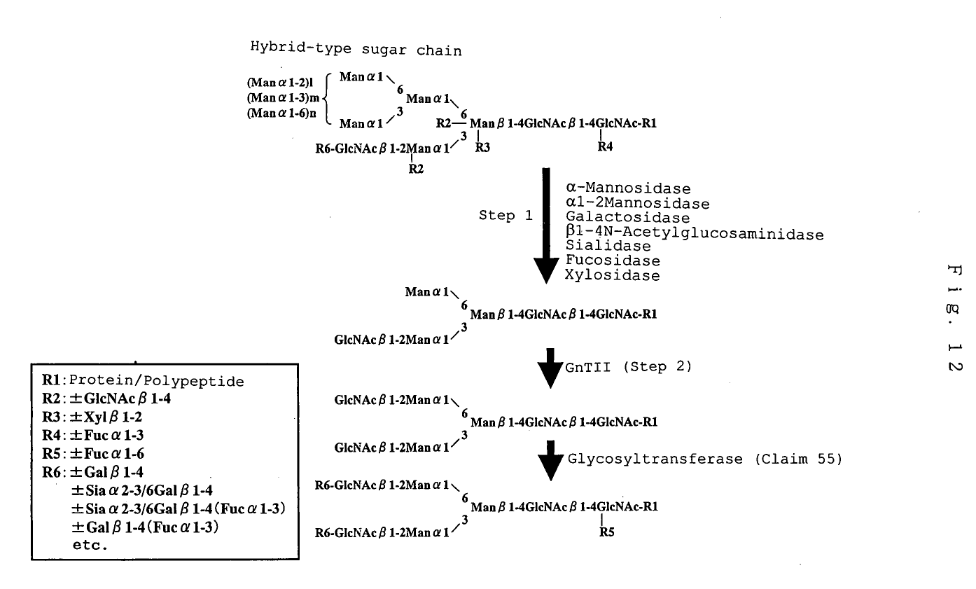

In step 1 of item 45, the sugar chain structure can be converted, for example, from a hybrid-type sugar chain or high mannose type sugar chain (see FIG. 11) to a Manα1-6(Manα1-3) Manα1-4GlcNAcβ1-4GlcNAc structure, and the glycosidase used in this step is preferably α-mannosidase. α-mannosidase is an enzyme that hydrolyzes mannose linked by the α1,2-linkage, α1,3-linkage, α1,4-linkage, or α1,6-linkage. Also, when xylose, fucose, N-acetylglucosamine, galactose, sialic acid, or the like is added to the converted glycoprotein sugar chain, then xylosidase, fucosidase, N-acetylglucosaminidase, galactosidase, sialidase, or the like which can hydrolyze the above can also be used with the α-mannosidase.

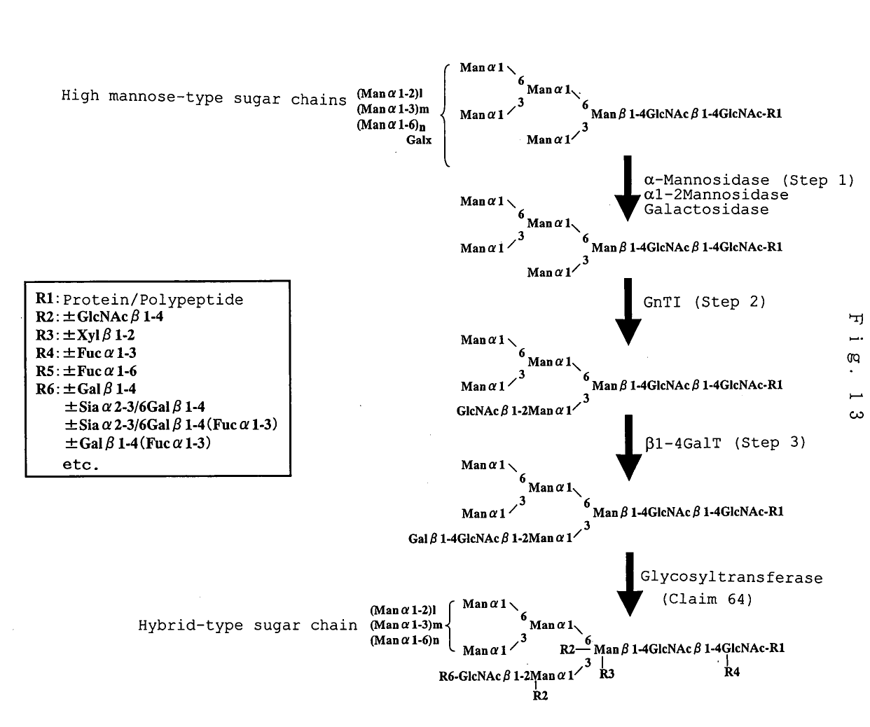

In step 1 of item 54, the sugar chain structure can be changed from, for example, a hybrid-type glycoprotein (see FIG. 12) to a Manα1-6(GlcNAcβ1-2Manα1-3)Manβ1-4GlcNAcβ1-4GlcNAc structure, but the glycosidase used in this step is preferably α1,2-mannosidase. α1,2-mannosidase is an enzyme that hydrolyzes mannose residues linked by the al, 2-linkage. Mannosidase I and Man₉-mannosidase are also included in α1,2-mannosidase in the present invention. When the outer chain of the converted glycoprotein sugar chain includes α1-3-linked mannose or α1-6-linked mannose, α-mannosidase can also be used. Furthermore, when xylose, fucose, N-acetyl glucosamine, galactose, sialic acid, or the like is included in sugar chains on the glycoprotein intended to convert its sugar chain, then at least one of selected from the group consisting xylosidase, fucosidase, N-acetylglucosaminidase, galactosidase, sialidase, or the like can also be used with the α-mannosidase. However, the β1,2-N-acetylglucosaminidase is not desirable because it will inhibit the formation of Manα1-6(GlcNAcβ1-2Manα1-3) Manβ1-4GlcNAcβ1-4GlcNAc, which is the target structure.

β1,2-N-acetylglucosaminyltransferase I (GnTI) is an enzyme that has the action of transferring GlcNAc via β1-2 linkages to the acceptor Manα1-6(Manα1-3)Manα1-6(Manα1-3)Manβ1-4GlcNAcβ1-4GlcNAc-R in the presence of UDP-GlcNAc as the sugar donor, so as to produce Manα1-6(Manα1-3)Manα1-6(GlcNAβ1-2Manα1-3)Manβ1-4GlcNAcβ1-4GlcNAc-R (where R is asparagine residue, peptide, protein, or a low- or high-molecular weight compound which does not inhibit the activity of other glycosyltransferases). It also transfers GlcNAc via β1-2 linkages to Manα1-6(Manα1-3)Manβ1-4GlcNAcβ1-4GlcNAc-R or Manα1-6(Manα1-2Manα1-3)Manα1-6(Manα1-3)Manβ1-4GlcNAc1-4GlcNAc-R.

β1,2-N-acetylglucosaminyltransferase II (GnTII) is an enzyme that has the action of transferring GlcNAc by means of β1-2 linkages to the acceptor Manα1-6(GlcNAcβ1-2Manα1-3)Manβ1-4GlcNAcβ1-4GlcNAc-R using at least UDP-GlcNAc as the sugar donor, so as to produce GlcNAcβ1-2Manα1-6(GlcNAcβ1-2Manα1-3)Manβ1-4GlcNAcβ1-4GlcNAc-R (where R is asparagine residue, peptide, protein, or a low- or high-molecular weight compound which does not inhibit the activity of other glycosyltransferases).

Glycoproteins with a converted sugar chain structure are easier to separate from the reaction system when at least one of the α1,2-mannosidase, β1,2-N-acetyl glucosaminyltransferase I, α-mannosidase, or β1,2-N-acetylglucosaminyltransferase II used in the invention is an immobilized enzyme.

In step 1 of item 24, glycoproteins having high mannose type sugar chains or hybrid-type sugar chains can be hydrolyzed under the following conditions. It will be apparent to those having ordinary skill in the art that glycoproteins having other sugar chains can also be hydrolyzed under appropriate conditions with reference to these conditions. It is shown in FIG. 9 that the sugar chain structure can be converted from high mannose type sugar chains or the hybrid-type sugar chains to Manα1-6(Manα1-3) Manα1-6(Manα1-3)Manα1-4GlcNAcα1-4GlcNAc, for example. In this step, the conversion can be carried out by allowing a suitable amount of a glycosidase such as α1,2-mannosidase to act for about 1 to 72 hours on a glycoprotein containing high mannose type sugar chains at a temperature from room temperature to about 40° C. in buffer with a pH of about 5 to 7 such as acetate buffer (pH 5.0) or MES buffer (pH 6.5). Alternatively, endoplasmic reticulum mannosidase can be allowed to act first prior to the action of the (α1,2-mannosidase. α-mannosidase or galactosidase can also be allowed to act first in cases of the sugar chais including α1-3 mannose residue, α1-6 mannose residue, or galactose residue.

In this step, the conversion can also be carried out by allowing a suitable amount of a (α1,2-mannosidase to act for about 1 to 72 hours on a glycoprotein containing hybrid-type sugar chains at a temperature from room temperature to about 40° C. in buffer with a pH of about 5 to 7 such as acetate buffer (pH 5.0) or MES buffer (pH 6.5). Alternatively, endoplasmic reticulum mannosidase can be allowed to act first prior to the action of the α1,2-mannosidase. α-mannosidase, sialidase, galactosidase, xylosidase, fucosidase, N-acetyl glucosaminidase, or the like can also be allowed to act first in cases of the sugar chains including α1-3 mannose residues, α1-6 mannose residue, sialic acid residue, galactose residue, xylose residue, fucose residue, N-acetylglucosamine residue, or the like.

The target product can be purified by dialysis after the completion of the reaction, but the target material is more readily purified in the case of using an immobilized enzyme such as immobilized α1,2-mannosidase.

Step 2 of item 24 can be carried out by allowing a suitable amount of β1,2-N-acetylglucosaminyltransferase I, in the presence of an equimolar or excess amount of UDP-GlcNAc, to act for about 1 to 72 hours at room temperature to about 40° C. on the glycoprotein obtained in step 1, in buffer with a pH of about 5 to 7 such as acetate buffer (pH 5.0) or MES buffer (pH 6.5). It is required for glycosylation by β1,2-N-acetylglucosaminyltransferase I that no sugar residue is linked to the non-reducing end of the Manα1-3Manβ1-4 branch of the sugar chain on the glycoprotein. The sugar chain structure Manα1-6(Manα1-3)Manα1-6(Manα1-3)Manβ1-4 containing of 5 mannose residues is an ideal substrate for β1,2-N-acetylglucosaminyltransferase I. However, the sugar chain structure Manα1-6(Manα1-3)Manβ1-4 containing of 3 mannose residues found in step 2 of item 45 is a more desirable substrate for β1,2-N-acetylglucosaminyltransferase I. The 6-mannose residue containing sugar chain structure Manα1-6(Manα1-2Manα1-3)Manα1-6(Manα1-3)Manβ1-4 is also a substrate for β1,2-N-acetylglucosaminyltransferase I. The sugar chain structure Manα1-6Manα1-6(Manα1-3)Manβ1-4 and Manα1-3Manα1-6(Manα1-3)Manβ1-4 can also serve as substrates for β1,2-N-acetylglucosaminyltransferase I. After the mannosidase treatment process in (1) above, it is no matter that the high mannose type sugar chains of the glycoproteins obtained have 3, 4, or 6 mannosidase residues. The target glycoprotein can be purified by dialysis or the like.

The target material can also be more readily purified with the use of immobilized β1,2-N-acetyl glucosaminyltransferase I.

In step 3 of item 24, Manα1-6(Manα1-3)Manα1-6(GlcNAcβ1-2Manα1-3)Manβ1-4GlcNAcβ1-4GlcNAc (see FIG. 9), for example, can be converted to the GnTII substrate Manα1-6(GlcNAcβ1-2Manα1-3)Manβ1-4GlcNAcβ1-4GlcNAc. This step can be carried out by allowing a suitable amount of α-mannosidase to act on the glycoprotein obtained in step 2, in a buffer with a pH of about 5 to 8, acetate buffer (pH 5.0), MES buffer (pH 6.5), and Tris-HCl buffer (pH 7.5). Examples of commercially available α-mannosidase include α-mannosidase derived from jack beans and α-mannosidase derived from almonds, and purified α-mannosidase or α-mannosidase II extracts from other organism sources can also be used.

Human mannosidase II isozymes or homologue genes which have been reported may also be used. Bacterial cell-derived mannosidase that hydrolyse α-1,2/3 linkages and α-1,6 linkages is commercially available (such as from New England Biolab). These may be used in combination.

Although the target glycoprotein can be purified by dialysis or the like, the target material is more readily purified in the case of immobilized α-mannosidase.

In step 4 of item 24, a suitable amount of β1,2-N-acetylglucosaminyltransferase II, in the presence of an equimolar or excess amount of UDP-GlcNAc, are allowed to act for about 1 to 72 hours at room temperature to about 40° C. on the glycoprotein obtained in step 3, in buffer with a pH of about 5 to 7 such as acetate buffer (pH 5.0) or MES buffer (pH 6.5), and the resulting material is purified by dialysis or the like, allowing the GlcNAc to be linked to the glycoprotein via β1,2-linkage. The target material can also be more readily purified in the case of immobilized β1,2-N-acetylglucosaminyltransferase I.

It is also possible to act galactosyltransferase (such as β1,3-galactosyltransferase or β1,4-galactosyltransferase) in the presence of UDP-Gal, sialyltransferase (such as α2,6-sialyltransferase or α2,3-sialyltransferase) in the presence of CMP-Sia, or fucosyltransferase (such as (α1,2-fucosyltransferase, α1,3-fucosyltransferase, α1,4-fucosyltransferase, or α1,6-fucosyltransferase) in the presence of GDP-Fuc on the glycoprotein obtained in the manner described above.

The target material is also more readily purified in the case of an immobilized glycosyltransferase. The glycosyltransferase used in the present invention may be produced naturally or by genetically engineering.

Step 1 in item 36 can be carried out in the same manner as step 2 in item 24 except that a recombinant glycoprotein is used instead of the glycoprotein obtained in step 1 of item 24.

Step 2 of item 36 can be carried out in the same manner as step 3 in item 24 using the glycoprotein obtained in step 1 of item 36 instead of the glycoprotein obtained in step 2 of item 24.

Step 3 of item 36 can be carried out in the same manner as step 4 in item 24 using the glycoprotein obtained in step 2 of item 36 instead of the glycoprotein obtained in step 3 of item 24.

It is also possible to act galactosyltransferase (such as β1,3-galactosyltransferase or β1,4-galactosyltransferase) in the presence of UDP-Gal, sialyltransferase (such as α2,6-sialyltransferase or α2,3-sialyltransferase) in the presence of CMP-Sia, or fucosyltransferase (such as α1,2-fucosyltransferase, α1,3-fucosyltransferase, α1,4-fucosyltransferase, or α1,6-fucosyltransferase) in the presence of GDP-Fuc on the glycoprotein obtained in the manner described above.

The target material is also more readily purified in the case of an immobilized glycosyltransferase. The glycosyltransferase used in the present invention may be produced naturally or by genetically engineering.

In step 1 of item 45, the glycoprotein can be converted, for example, from high mannose type sugar chains or hybrid-type sugar chains to Manα1-3(Manα1-6)Manβ1-4GlcNAcβ1-4GlcNAc (see FIG. 11). This step can be carried out by allowing a suitable amount of α-mannosidase to act on the glycoprotein obtained in step 2, in a buffer with a pH of about 5 to 7, such as acetate buffer (pH 5.0) or MES buffer (pH 6.5). Sialidase, galactosidase, xylosidase, fucosidase, N-acetylglucosaminidase, or the like can also be allowed to act first in cases of sugar chain on glycoprotein including sialic acid residue, galactose residue, xylose residue, fucose residue, N-acetylglucosamine residue, or the like.

The target product can be purified by dialysis after the conclusion of the reaction, but the target material is more readily purified in the case of an immobilized enzyme such as immobilized α-mannosidase.

Step 2 of item 45 can be carried out in the same manner as step 2 in item 24 using the glycoprotein obtained in step 1 of item 45 instead of the glycoprotein obtained in step 1 of item 24.

Step 3 of item 45 can be carried out in the same manner as step 4 in item 24 using the glycoprotein obtained in step 2 of item 45 instead of the glycoprotein obtained in step 3 of item 24.

It is also possible to act galactosyltransferase (such as β1,3-galactosyltransferase or β1,4-galactosyl transferase) in the presence of UDP-Gal, sialyltransferase (such as α2,6-sialyltransferase or α2,3-sialyltransferase) in the presence of CMP-Sia, or fucosyltransferase (such as α1,2-fucosyltransferase, α1,3-fucosyltransferase, α1,4-fucosyltransferase, or α1,6-fucosyltransferase) in the presence of GDP-Fuc on the glycoprotein sugar chains obtained in the manner described above.

The target material is also more readily purified in the case of an immobilized glycosyltransferase. The glycosyltransferase used in the present invention may be produced naturally or by genetically engineering.

In step 1 of item 54, hybrid-type sugar chains (see FIG. 12) on glycoproteins, for example, can be converted to a Manα1-3(GlcNAcβ1-2Manα1-6)Manβ1-4GlcNAcβ1-4GlcNAc structure. This step can be carried out in the same manner as step 1 in item 45 except that only glycoproteins with hybrid-type sugar chains are used in step. 1 of item 45, and no β1,2-N-acetylglucosaminidase is used as the glycosidase.

Step 2 of item 54 can be carried out in the same manner as step 3 in item 45 using the glycoprotein obtained in step 1 of item 54 instead of the glycoprotein obtained in step 2 of item 45.

Item 63 is a method for converting the high mannose type sugar chains on glycoproteins to hybrid-type sugar chains using an immobilized enzyme in at least one of steps 1 through 3.

Step 1 in item 63 can be carried out in the same manner as step 1 in item 24 except that the sugar chains of the glycoprotein that is used are only high mannose types.

Step 2 of item 63 can be carried out in the same manner as step 2 in item 24 using the glycoprotein obtained in step 1 of item 63 instead of the glycoprotein obtained in step 1 of item 24.

Step 3 of item 63 can be carried out by allowing a suitable amount of β1,4-galactosyltransferase I in the presence of an equimolar or excess amount of UDP-Gal to act for about 1 to 72 hours at room temperature to about 40° C. on the glycoprotein obtained in step 2 of item 63, in buffer with a pH of about 6 to 9 such as MES buffer (pH 6.5) or Tris-HCl buffer (pH 8.0). The target product can be purified by dialysis after the completion of the reaction, but the target material is more readily purified in the case of immobilized β1,4-galactosyltransferase.

It is also possible to act galactosyltransferase (such as β1,3-galactosyltransferase or β1,4-galactosyl transferase) in the presence of UDP-Gal, sialyltransferase (such as α2,6-sialyltransferase or α2,3-sialyltransferase) in the presence of CMP-Sia, or fucosyltransferase (such as α1,2-fucosyltransferase, α1,3-fucosyltransferase, α1,4-fucosyltransferase, or α1,6-fucosyltransferase) in the presence of GDP-Fuc on the glycoprotein sugar chains obtained in the manner described above.

The target material is also more readily purified with the use of an immobilized glycosyltransferase for the glycosyltransferase in this case.

The glycosyltransferase used in the present invention may be produced naturally or by genetically engineering.

### BEST MODE FOR CARRYING OUT THE INVENTION

The present invention is illustrated in further detail in the following examples, but these are examples only and do not in any way limit the scope of the invention.

### EXAMPLE 1

Obtaining hGnTIIcDNA Fragments by PCR

The oligonucleotides represented in SEQ ID NO. 4 and 5 were synthesized based on the nucleotide sequence for the human β1,2-N-acetylglucosaminyltransferase II (hGnTII) cDNA in SEQ ID NO. 1. The former is the nucleotide sequence 4-27 in the nucleotide sequence in SEQ ID NO. 1, and the latter is the nucleotide sequence 1341-1377 in the nucleotide sequence in SEQ ID NO. 1. These oligonucleotides were used as primers in PCR under the following conditions using a Caucasian female placenta chromosomal DNA (Clontech) as template.

{1 μL of 0.1 mg/mL Caucasian female placenta chromosomal gene, 1 μL each of 10 pmole/μL primer, 0.5 μL Taq. DNA Polymerase (Takara PCR Kit, by Takara Shuzo), 5 μL of 10-fold concentrated PCR buffer (Takara PCR Kit, by Takara Shuzo), 4 μL dNTP (Takara PCR Kit, by Takara Shuzo), 37.5 μL sterilized water, total reaction solution was 50 μL, reaction temperature (time): denaturation 94° C. (2 min), annealing 50° C. (2 min), elongation 72° C. (1 min); number of cycles: 30.}

After the PCR reaction, the reaction solution was applied to agarose gel electrophoresis to check the DNA fragment (PCR product). The DNA fragment was ligated to the pGEM-Teasy (Promega) vector with T4 DNA ligase according to TA cloning method, E. coli JM109 was transformed, and transformants were selected on LB plate media containing 50 μg/mL ampicillin, IPTG, and X-gal.

Plasmids with the DNA fragments were prepared from the E. Coli, and the DNA fragment nucleotides were sequenced in the usual manner, revealing that the sequence was consistent with the nucleotide sequence 4-1377 in the nucleotide sequence of SEQ ID NO. 1. It was thus confirmed that the desired DNA coding for hGnTII had been obtained.

### EXAMPLE 2

Construction of Expression Vector pMGNT-II

The oligonucleotides in SEQ ID NOS. 6 and 7 were synthesized based on the nucleotide sequence for β1,2-N-acetyl glucosaminyltransferase II (hGnTII) cDNA in SEQ ID NO. 1 in order to obtain DNA coding for the GnTII lacking the transmembrane domain. In the former, a BamHI recognition site was ligated to the 5′ terminal of the nucleotide sequence 85-105 in the nucleotide sequence of SEQ ID NO. 1, and in the latter, a HindIII recognition site was ligated to the 3′ terminal of the nucleotide sequence 1334-1359 in the nucleotide sequence of SEQ ID NO. 1. These oligonucleotides were used as primers for PCR under the following conditions using the hGnTII cDNA on the pGEM-Teasy obtained in Example 1 as template.

{1 μL of 0.1 mg/mL hGnTII cDNA obtained in Example 1, 1 μL each of 10 pmole/μL primer, 0.5 μL Taq. DNA Polymerase (Takara PCR Kit, by Takara Shuzo), 5 μL of 10-fold concentrated PCR buffer (Takara PCR Kit, by Takara Shuzo), 4 μL dNTP (Takara PCR Kit, by Takara Shuzo), 37.5 μL sterilized water, total reaction soulution is 50 μL, reaction temperature (time): denaturation 94° C. (2 min), annealing 50° C. (2 min), elongation 72° C. (1 min); number of cycles: 30.}

The PCR products were digested with the BamHI and HindIII restriction enzymes, and approximately 1.2 kb fragments were recovered from electrophoresed agarose gel. Meanwhile, the pMAL-c2 plasmid (New England Biolabs) was digested with the BamHI and HindIII restriction enzymes, and approximately 6.6 kb fragments were recovered from electrophoresed agarose gel. The two fragments were ligated using T4 DNA ligase, giving the novel plasmid pMGNT-II.

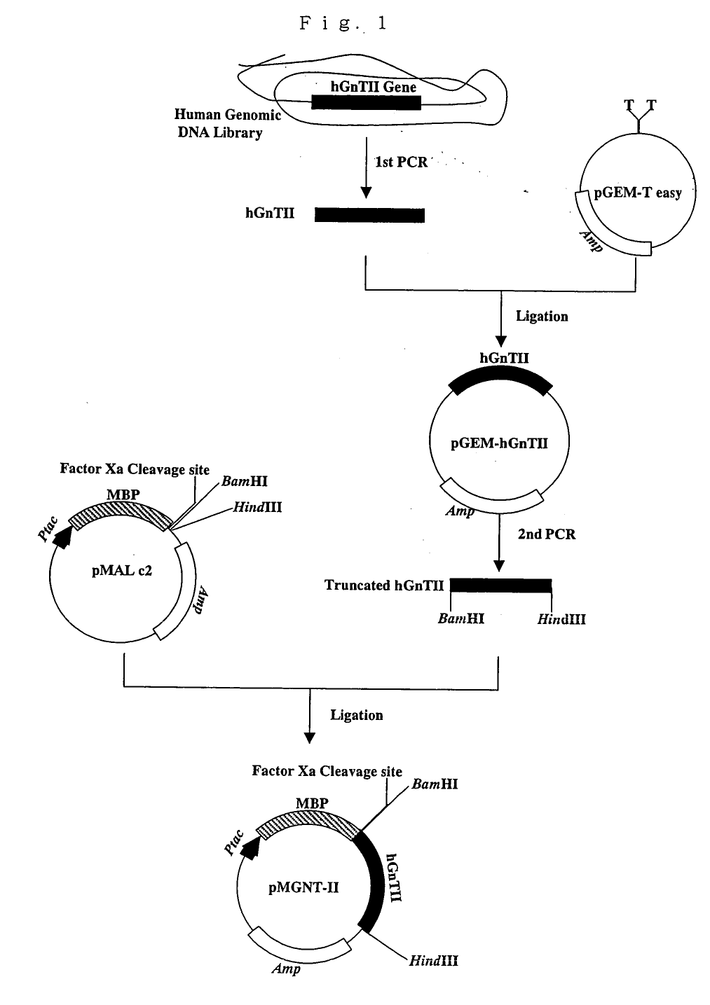

The above procedure is summarized in FIG. 1.

### EXAMPLE 3

Preparation of MBP-hGnTII Fusion Protein Using Recombinant E. coli

E. coli DH5α was transformed in the usual manner with the pMGNT-II expression vector obtained in Example 2. Two test tubes filled with 5 mL of LB medium containing 50 μg/mL ampicillin were inoculated with 1 platinum loop of the resulting recombinant E. coli for shaking culture overnight at 37° C. 10 mL of the resulting culture was used to inoculate culture flasks filled with 1 L of the above medium. When the optical density of the culture at 610 nm reached 0.5 during shaking culture at 37° C., IPTG was added to a final concentration of 1 mM, and the cultivation was continued for another 4 hours at 37° C.

The cells were harvested by centrifugation and resuspended in 23 mL of 100 mM MES buffer (pH 6.5, containing 20 mM MnCl₂, 50 mM NaCl, and 1 mM 2-mercaptoethanol). The suspension was treated with a sonicator and centrifuged, giving supernatant in the form of a crude enzyme solution. The crude enzyme solution was allowed to flow through an amylose resin (New England Biolabs) column (5 mL) which had been previously equilibrated with the above buffer, thereby allowing the MBP-hGnTII fusion protein to be adsorbed. The column was washed with the above buffer, and the adsorbed fusion protein was eluted with the above buffer containing 10 mM maltose. The eluted fractions were collected, and the resulting fusion protein was dialyzed against the above buffer, giving a fusion protein solution(MBP-hGnTII).

### EXAMPLE 4

Assay of GnTII Activity

A reaction was carried out for 3 hours at 37° C. between 23 μL of the fusion protein (MBP-hGnTII) obtained in Example 3, 1 μL of 308 mM uridine-5′-diphospho-N-acetylglucosamine (UDP-GlcNAc), and reaction solution containing 1 μL of 10 μM pyridylaminated oligosaccharide (Takara Shuzo) represented by Formula 2 below.

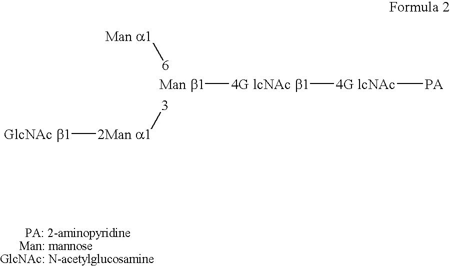

After the reaction, the reaction was stopped by 2 minutes of incubation in boiling water. Analysis of the reaction solution by HPLC revealed a product (Formula 3) to which N-acetylglucosamine had been transferred.

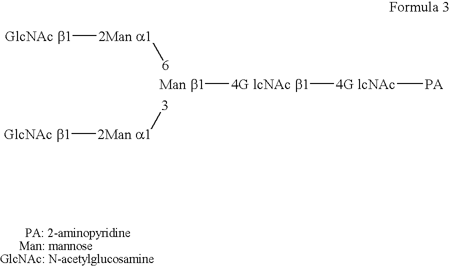

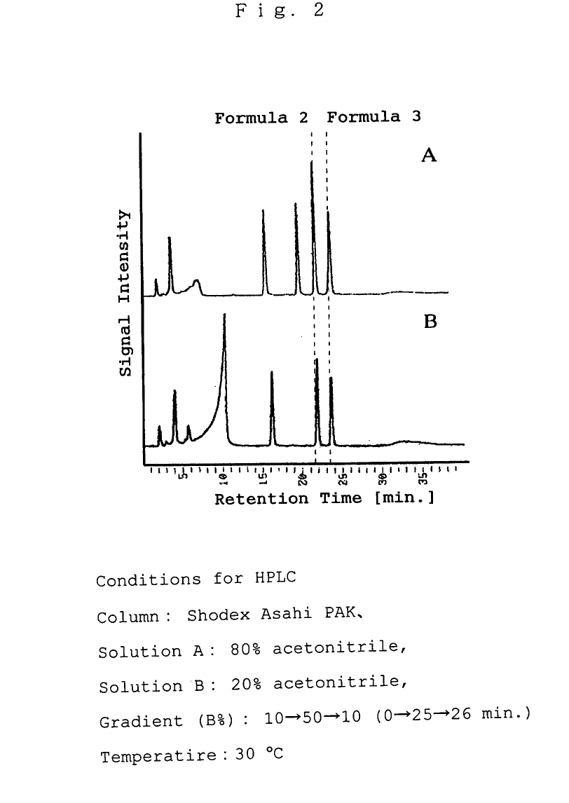

The results are given in FIG. 2. FIG. 2A gives the results of analysis for the standard pyridylaminated oligosaccharides (Takara Shuzo) represented by Formulas 2 and 3, and FIG. 2B gives the results from 3 hours of reaction using the pyridylaminated oligosaccharide represented by Formula 2 as the substrate. The peak for the pyridylaminated oligosaccharide represented by Formula 3 in FIG. 2B was produced by MBP-hGnTII.

The above results confirmed that the MBP-hGnTII obtained by the method of the invention had GnTII enzyme activity.

### EXAMPLE 5

Study on hGnTII Substrate Specificity

Reaction was carried out overnight at 37° C. in 50 μL of reaction solution containing the MBP-hGnTII obtained in Example 3 (23 μL), 308 mM UDP-GlcNAc (1 μL), and 1 μL of 10 μM pyridylaminated oligosaccharides represented by Formulas 2 and 4-8. After the reaction, the reaction was stopped by 2 minutes of incubation in boiling water. Analysis of the reaction solution by HPLC in the same manner as in Example 4 revealed activity only for the pyridylaminated oligosaccharide of Formula 6 other than the pyridylaminated oligosaccharide of Formula 2 (Table 1) The above results confirmed that the MBP-hGnTII obtained by the method of the invention had the same substrate specificity as the natural one.

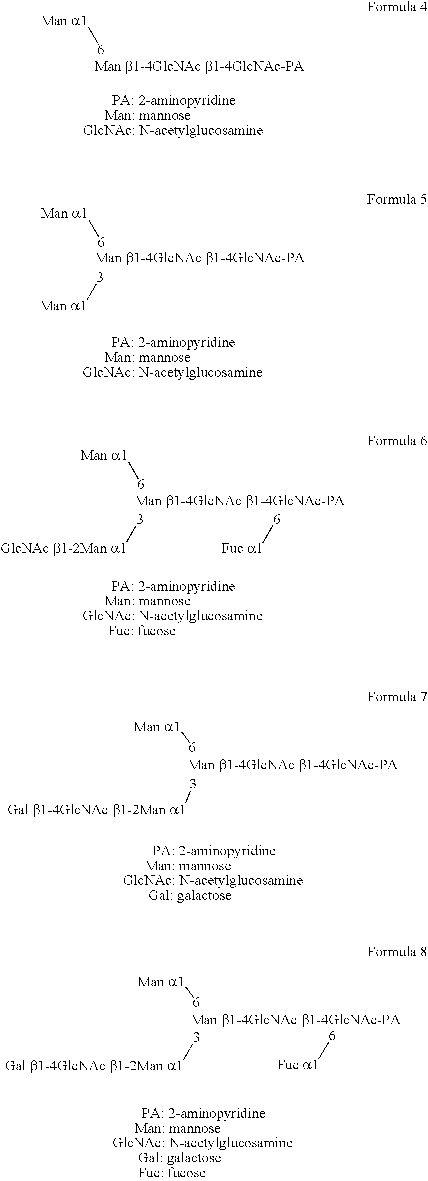

TABLE 1

| Sugar Chain Substrate | GnTII activity |
|---|---|
| Formula 2 | + |
| Formula 4 | − |
| Formula 5 | − |
| Formula 6 | + |
| Formula 7 | − |
| Formula 8 | − |

### EXAMPLE 6

Study on Optimal Reaction Temperature for MBP-hGnTII

Reaction was carried out for 3 hours at temperatures from 0 to 70° C. in 50 μL of reaction solution containing the MBP-hGnTII obtained in Example 3 (23 μL), 308 mM UDP-GlcNAc (1 μL), and 1 μL of 10 μM pyridylaminated oligosaccharide represented by Formula 2. After the reaction, the reaction was stopped by 2 minutes of incubation in boiling water. The reaction solutions were analyzed by HPLC in the same manner as in Example 4 to assay GnTII activity and the optimal temperature reaction for the MBP-hGnTII.

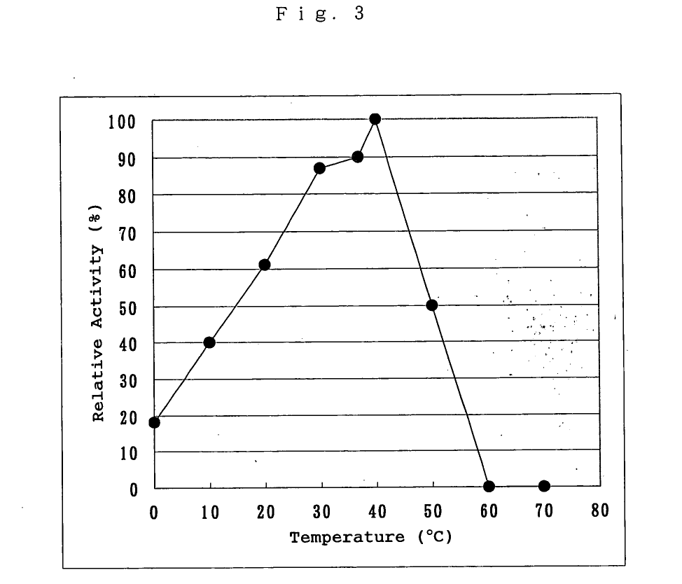

The optimal reaction temperature was found to be between 30 and 40° C. (FIG. 3).

### EXAMPLE 7

Study on Thermal Stability of MBP-hGnTII

The MBP-hGnTII obtained in Example 3 (23 μL) was incubated for 4 hours at temperatures between 0 and 70° C., and was then cooled on ice for 5 minutes. 308 mM UDP-GlcNAc (1 μL) and 1 μL of 10 μM pyridylaminated oligosaccharide represented by Formula 2 were added, and a reaction was carried out for 5 hours at 37° C.

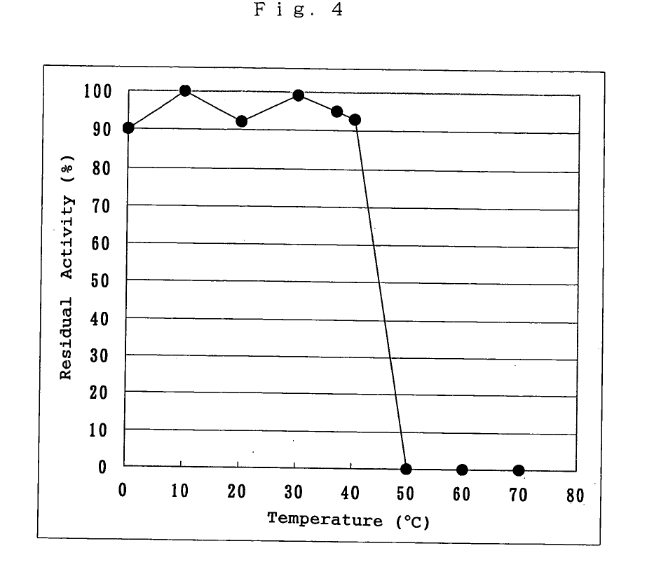

After the reaction, the reaction was stopped by 2 minutes of incubation in boiling water. The reaction solutions were analyzed by HPLC in the same manner as in Example 4 to assay GnTII activity and the thermal stability of the MBP-hGnTII. The results confirmed that MBP-hGnTII was thermally stable up to 40° C. (FIG. 4).

### EXAMPLE 8

Study on Optimal pH for MBP-hGnTII

The MBP-hGnTII obtained in Example 3 was dialyzed against 3 mM MES buffer (pH 6.5, containing 24 mM MnCl₂, 60 mM NaCl, 1.2 mM 2-mercaptoethanol) in order to lower the buffer concentration. 5 μL each of 200 mM buffer solutions with pH levels ranging from 4.0 to 10.5, 1.5 μL of the 10 μM pyridylaminated oligosaccharide represented by Formula 2, and 308 mM UDP-GlcNAc (1 μL) were added to the resulting MBP-hGnTII (25 μL), and reactions were carried out for 5 hours at 37° C. After the reaction, the reaction was stopped by 2 minutes of incubation in boiling water. The reaction solutions were analyzed by HPLC in the same manner as in Example 4 to assay GnTII activity and the optimal pH for the MBP-hGnTII. The results confirmed that MBP-hGnTII was thermally stable up to 40° C. (FIG. 4). The used buffers were acetate buffer (pH 4.0 to 5.5), cacodylate buffer (pH 5.5 to 7.0), HEPES buffer (pH 7.0 to 8.0), Tricine buffer (pH 8.0 to 8.8), bicine buffer (pH 8.0 to 8.8), and glycine buffer (pH 8.8 to 10.5).

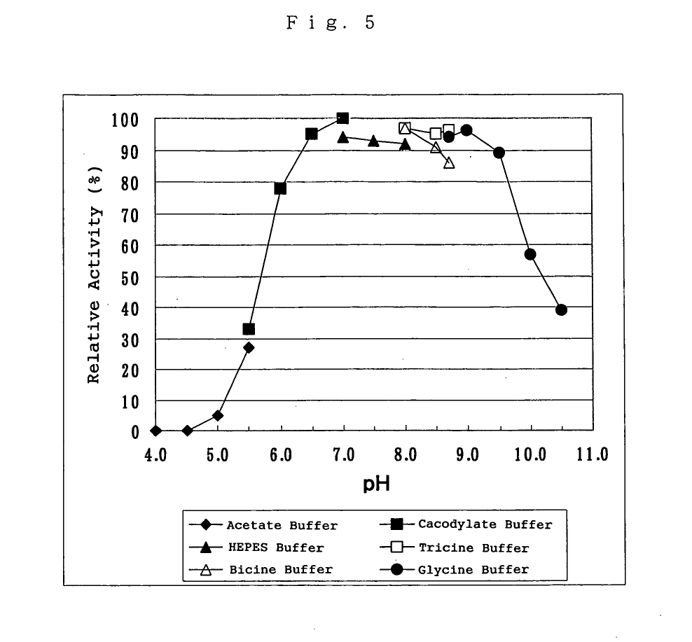

The results confirmed that the optimal pH for MBP-hGnTII is a pH of 6.5 to 9.0 (FIG. 5).

### EXAMPLE 9

Study on pH Stability of MBP-hGnTII

Following the same dialysis as in Example 8, 5 μL each of 200 mM buffer solutions with pH levels of 4.0 to 10.5 were added to the resulting MBP-hGnTII (25 μL), and the resulting solutions were incubated for 7 hours at 25° C. 7.5 μL of 1 M MES buffer (pH 6.5, containing 20 mM MnCl₂, 50 mM NaCl, and 1 mM 2-mercaptoethanol), 2 μL of 10 μM pyridylaminated oligosaccharide represented by Formula 2, and 308 mM UDP-GlcNAc (1 μL) were then added, and a reaction was carried out for 5 hours at 37° C. After the reaction, the reaction was stopped by 2 minutes of incubation in boiling water. The reaction solutions were analyzed by HPLC in the same manner as in Example 4 to assay GnTII activity and the pH stability of the MBP-hGnTII. The used buffers were acetate buffer (pH 4.0 to 5.5), cacodylate buffer (pH 5.5 to 7.0), HEPES buffer (pH 7.0 to 8.0), Tricine buffer (pH 8.0 to 8.8), bicine buffer (pH 8.0 to 8.8), and glycine buffer (pH 8.8 to 10.5).

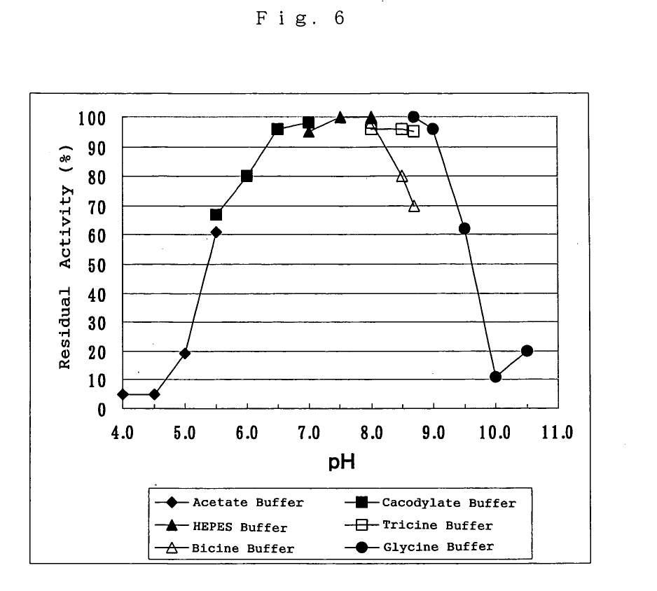

The results confirmed that the MBP-hGnTII was stable at a pH between 5.5 and 9.5 (FIG. 6).

### EXAMPLE 10

Conversion of RNaseB With High Mannose Sugar Chains to RNaseB With Complex-Type Sugar Chains

(1) Sugar Chain Conversion Reaction

Step 1: α1,2-Mannosidase Digestion

20 mg of RNaseB was dissolved in 360 μL of 20 mM acetate buffer (pH 5.0), 20 μL of 0.5 U/mL α1,2-mannosidase was added, and a reaction was carried out for 18 hours at 37° C. 15 μL of 0.5 U/mL α1,2-mannosidase was again added thereto, and a reaction was carried out for another 9 hours at 37° C. 5 μL of 0.5 U/mL α1,2-mannosidase was again added thereto, and a reaction was carried out for another 18 hours at 37° C.

300 μL out of the resulting reaction solution was dialyzed against 100 mM MES buffer (pH 6.5, containing 20 mM MnCl₂, 50 mM NaCl, and 1 mM 2-mercaptoethanol). The remaining 100 μL was dialyzed against distilled water for use as a sample to analyze the sugar chain conversion.

Step 2: β1,2-N-acetylglucosaminyltransferase I Reaction

60 μL of 308 mM UDP-GlcNAc and 660 μL maltose-binding protein-β1,2-N-acetylglucosaminyltransferase I fusion protein (MBP-hGnTI) were added to the α1,2-mannosidase-digested RNaseB (280 μL) obtained in step 1, and a reaction was carried out for 18 hours at 37° C.

666 μL of the resulting reaction solution was dialyzed against 10 mM MES buffer (pH 6.0). The remaining 334 μL was dialyzed against distilled water for use as a sample to analyze the sugar chain conversion.

The MBP-hGnTI obtained by the method in Japanese Unexamined Patent Application (Kokai) 2001-178453 was used.

Step 3: Jack Bean α-Mannosidase Digestion

94 μL of 100 mM MES buffer (pH 6.0) and 94 μL of Jack bean α-mannose (by Seikagaku Kogyo) were added to the MBP-hGnTI treated RNaseB (750 μL) obtained in step 2, and a reaction was carried out for 15.5 hours at 37° C. After the reaction, another 47 μL of Jack bean α-mannosidase was added, and a reaction was carried out for 13.5 hours at 37° C. After the reaction, another 47 μL of the above α-mannosidase was added, and a reaction was carried out for 10.5 hours at 37° C. After the reaction, another 94 μL of the above α-mannosidase was added, and a reaction was carried out for 7 hours at 37° C. After the reaction, another 94 μL of the above α-mannosidase was added, and a reaction was carried out for 43.5 hours at 37° C., for a total of 90 hours of reaction.

610 μL out of the resulting reaction solution was dialyzed against 100 mM MES buffer (pH 6.5, containing 20 mM MnCl₂, 50 mM NaCl, and 1 mM 2-mercaptoethanol). The remaining 610 μL was dialyzed against distilled water for use as a sample to analyze the sugar chain conversion.

Step 4: β1,2-N-acetylglucosaminyltransferase II Reaction

60 μL of 308 mM UDP-GlcNAc and 2.19 mL of the MBP-hGnTII obtained in Example 3 were added to the Jack bean α-mannosidase digested RNaseB (750 μL) obtained in step 3, and a reaction was carried out for 40 hours at 37° C. After the reaction, another 1 mL of MBP-hGnTII was added, and a reaction was carried out for another 11 hours at 37° C. After the reaction, another 1 mL of MBP-hGnTII was added, and a reaction was carried out for another 12.5 hours at 37° C. After the reaction, another 300 μL of MBP-hGnTII was added, and a reaction was carried out for another 11 hours at 37° C. After the reaction, another 500 μL of MBP-hGnTII was added, and a reaction was carried out for another 10.5 hours at 37° C., for a total of 85 hours of reaction.

The resulting reaction solution was dialyzed against distilled water to analyze the sugar chain conversion.

(2) Analysis of Sugar Chain Conversion

Sugar chains were cut out in the usual manner from samples obtained in steps 1-4 above for fluorescent labeling with 2-aminopyridine. The resulting pyridylaminated oligosaccharides were analyzed by normal phase HPLC.

The conditions for the HPLC are given below.

Column: Amide column Shodex Asahi PAK,

Soln. A: 80% CH₃CN,

Soln. B : 20% CH₃CN,

Gradient : 10→50→10% Soln. B (5→25→26 min),

Time : 40 min,

Column temp.: 30 ° C.

(a) Analysis of the product in Step 1

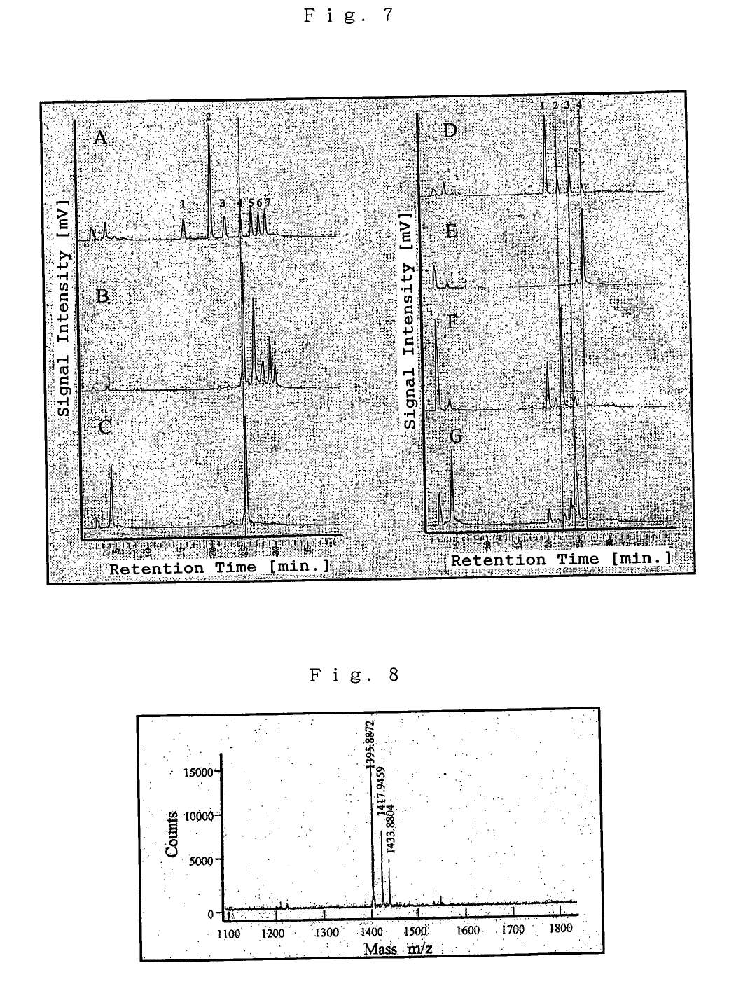

FIG. 7A shows the results of analysis for the standard pyridylaminated oligosaccharide. Peaks 1, 2, 3, 4, 5, 6, and 7 correspond to the pyridylaminated oligosaccharides represented by Formulas 4, 5, 9, 10, 11, 12, and 13, respectively.

The results of the sugar chain analysis for untreated RNaseB (FIG. 7B) confirmed the presence of various high mannose-type sugar chains corresponding to peaks 4-7 in FIG. 7a.

The results of sugar chain analysis of the α1,2-mannosidase digested RNaseB obtained in step 1 (FIG. 7C) confirmed that all of the peaks for the various high mannose sugar-type chains detected in FIG. 7B had been converted to peak 4 (Formula 10).

(b) Analysis of the product in Step 2

FIG. 7D also shows the results of analysis for the standard pyridylaminated oligosaccharide, where peaks 1, 2, 3, and 4 correspond to the pyridylaminated oligosaccharides of Formulas 5, 6, 3, and 14, respectively.

The results of analysis for the sugar chains of the RNaseB treated with the MBP-hGnTI obtained in step 2 (FIG. 7E) revealed only a peak corresponding to peak 4 (Formula 14) in FIG. 7D.

(c) Analysis of the product in Step 3

The results of analysis for the sugar chains of the Jack bean α-mannosidase digested RNaseB obtained in step 3 (FIG. 7F) confirmed that the peak corresponding to peak 2 (Formula 6) in FIG. 7D was the major reaction product.

(d) Analysis of the product in Step 4

The results of analysis for the sugar chains of the RNaseB treated with MBP-hGnTII obtained in step 4 (FIG. 7G) confirmed that the peak corresponding to peak 3 (Formula 3) in FIG. 7D was the primary reaction product.

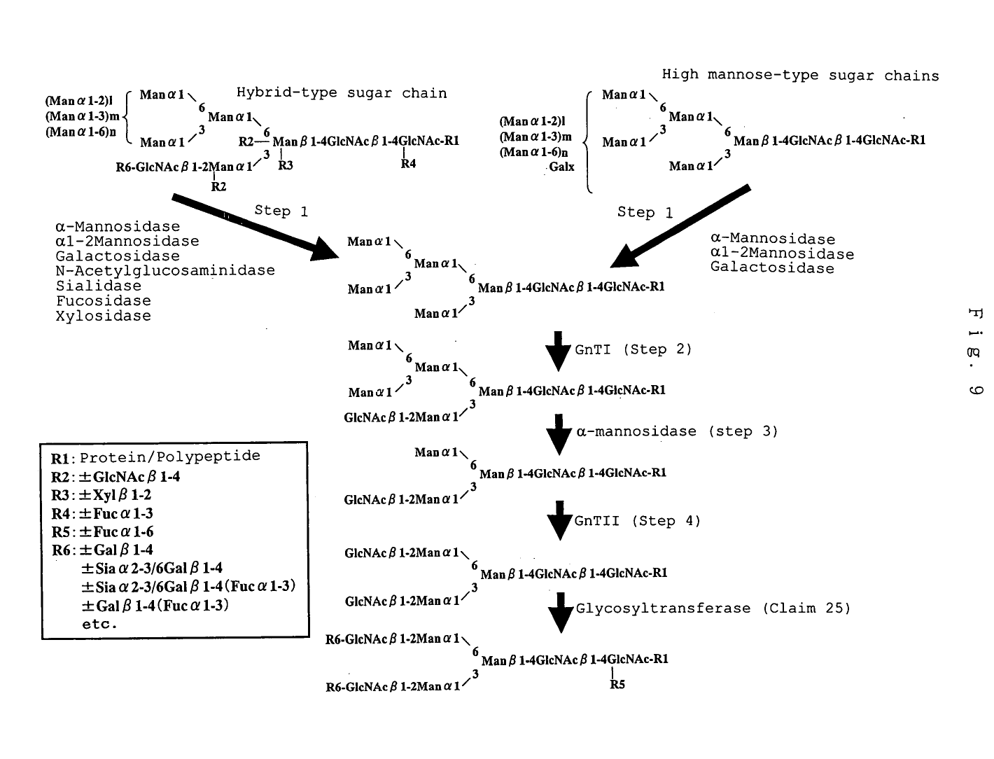

The results of mass spectrometry analysis of the fractionated main peak in FIG. 7G (FIG. 8) gave value virtually consistent with the theoretical value for the pyridylaminated oligosaccharide of Formula 3 (1395.32), thus confirming that the sugar chain structure of the RNaseB treated with the β1,2-N-acetylglucosaminyltransferase II obtained in step 4 had the structure represented by Formula 15.

The above results confirmed that RNaseB with various high mannose-type sugar chains could be readily converted enzymatically to RNaseB having complex-type sugar chains.

### EXAMPLE 11

Preparation of α-Mannosidase II

The livers of 30 mice were homogenized according to the method in J. Biol. Chem., 266:16876 (1991), Golgi body-rich membrane fraction was then obtained. α-mannosidase II was purified from the membrane fraction as follows: solubilization, phase separation in Triton X-114, digestion with α-chymotrypsin, phase separation in Triton X-114, and chromatography on a Mono S (Amersham Pharmacia) column and on a Superose 6 (Amersham Pharmacia) column. The resulting α-mannosidase II fraction was concentrated using Sentricon YM-30, giving 0.5 mL of α-mannosidase II solution.

### EXAMPLE 12

Conversion of RNaseB With High Mannose-Type Sugar Chains to RNaseB With Complex-Type Sugar Chains

RNaseB sugar chains were converted in the same manner as in Example 10 except that the α-mannosidase II obtained in Example 11 was used instead of the Jack bean α-mannosidase in step 3, and 100 mM MES buffer (pH 6.0) containing 0.1% Triton X-100 was used instead of 100 mM MES buffer (pH 6.0) in step 3. Analysis of the converted RNaseB sugar chains revealed a product corresponding to Formula 3.

### EXAMPLE 13

Preparation of Immobilized β1,2-N-Acetylglucosaminyltransferase II

In the same manner as in Example 3, the MBP-hGnTII fusion protein was adsorbed to an amylose resin (New England Biolabs) column (5 mL) and washed with 100 mM MES buffer (pH 6.5, containing 20 mM MnCl₂, 50 mM NaCl, and 1 mM 2-mercaptoethanol, and the resulting resin was used as such for immobilized enzyme.

### EXAMPLE 14

Preparation of Immobilized β1,2-N-Acetylglucosaminyltransferase I

Recombinant E. coli producing the MBP-hGnTI fusion protein as noted in Japanese Unexamined Patent Application (Kokai) 2001-178453 was used to obtain immobilized β1,2-N-acetylglucosaminyltransferase I (5 mL) in the same manner as in Example 11.

### EXAMPLE 15

Preparation of Immobilized α1,2-Mannosidase

0.5 U of α1,2-mannosidase (Seikagaku Kogyo) and 5 mL of 50 mM HEPES buffer (pH 7.0) containing 20 mg bovine serum albumin were added to 1 mL of NHS activated Sepharose 4FF (Amersham Pharmacia) which had been previously washed with 60 mL of 1 mM hydrochloric acid, and the contents were gently shaken overnight at 4° C. to ensure that the enzyme was immobilized. The resin was then filtered off and washed with 5 mL of 50 mM HEPES buffer (pH 7.0). The resin was then placed in 5 mL of 0.1 M Tris-HCl buffer (pH 8.0) and gently shaken for 4 hours at 4° C. to block the remaining activated sites in the resin. The resin was then washed with 1 M sodium chloride aqueous solution and distilled water, and 1 mL of the resulting immobilized α1,2-mannosidase was immersed in 50 mM acetate buffer (pH 5.0) and stored at 4° C.

### EXAMPLE 16

Preparation of Immobilized α-Mannosidase II

1 mL of immobilized α-mannosidase II was obtained in the same manner as in Example 15 except that 0.5 mL of the α-mannosidase II obtained in Example 11 was used instead of the 0.5 U of α1,2-mannosidase (Seikagaku Kogyo).

### EXAMPLE 17

Preparation of Immobilized β1,4-Galactosyltransferase

30 U of β1,4-galactosyltransferase (Toyobo), 120 mg of bovine serum albumin, and 10 mL of 50 mM HEPES buffer (pH 7.0) containing 1 mM of uridine-5′-diphosphogalactose (UDP-Gal), 5 mM N-acetylglucosamine, and 25 mM manganese chloride were added to 3 mL CNBr-activated Sepharose 4B (Amersham Pharmacia) which had been previously washed with 100 mL of 1 mM hydrochloric acid, and the contents were gently shaken overnight at 4° C. to ensure that the enzyme was immobilized. The resin was then filtered off and washed with 10 mL of 50 mM HEPES buffer (pH 7.0). The resin was then placed in 10 mL of 0.1 M Tris-HCl buffer (pH 8.0) and gently shaken for 4 hours at 4° C. to block the remaining activated sites in the resin. The resin was then washed with 1 M sodium chloride aqueous solution and distilled water, and 3 mL of the resulting immobilized β1,4-galactosyltransferase was immersed in 25 mM HEPES buffer (pH 7.0) containing UDP-Gal (1 mM) and 5 mM manganese chloride, and stored at 4° C.

### EXAMPLE 18

Preparation of Immobilized α2,6-Sialyltransferase

1.2 U of α2,6-sialyltransferase (Toyobo), 75 mg bovine serum albumin, and 10 mL of 50 mM HEPES buffer (pH 7.0) containing 1 mM cytidine-5′-diphosphate and 5 mM N-acetyllactosamine were added to 1.0 g of NHS activated Sepharose 4FF (Amersham Pharmacia) which had been previously washed with 100 mL of 1 mM hydrochloric acid, and the contents were gently shaken overnight at 4° C. to ensure that the enzyme was immobilized. The resin was then filtered off and washed with 10 mL of 50 mM HEPES buffer (pH 7.0). The resin was then placed in 10 mL of 0.1 M Tris-HCl buffer (pH 8.0) and gently shaken for 4 hours at 4° C. to block the remaining activated sites in the resin. The resin was then washed with 1 M sodium chloride aqueous solution and distilled water, and 3 mL of the resulting immobilized α2,6-sialyltransferase was immersed in 25 mM HEPES buffer (pH 7.0) containing 1 mM cytidine-5′-monophospho-N-acetylneuraminic acid (CMP-NeuAc), and stored at 4° C.

### EXAMPLE 19

Preparation of Immobilized α-Mannosidase

Immobilized α-mannosidase was obtained in the same manner as in Example 11 using 30 U α-mannosidase (from Jack beans, by Seikagaku Kogyo), and was immersed in 50 mM HEPES buffer (pH 7.0) containing 0.01 mM zinc acetate, and stored at 4° C.

### EXAMPLE 20

Use of Immobilized Enzyme for Conversion of RNaseB High Mannose-Type Sugar Chains to Hybrid-Type Sugar Chains

(1) Sugar Chain Conversion Reaction

Step 1: Trimming High Mannose-Type Sugar Chains With Immobilized α1,2-Mannosidase

100 mg RNaseB (Sigma) was dissolved in 2 mL of 20 mM acetate buffer (pH 5.0), 1 mL of the immobilized α1,2-mannosidase obtained in Example 15 was added thereto, and a reaction was carried out as the contents were gently shaken for 24 hours at 37° C. After the reaction, the immobilized 1,2-mannosidase was filtered off, and the immobilized α1,2-mannosidase was washed with 5 mL of distilled water. The reaction solution and the wash were combined, the mixture was introduced into a dialysis membrane (by Spectrum) with a 3.5 kD molecular weight cut off and was dialyzed against distilled water, and it was then lyophilized, giving RNaseB (87 mg) in which the target sugar chains had been trimmed.

Step 2: Elongation of Sugar Chains with Immobilized β1,2-N-acetylglucosaminyltransferase I

The RNaseB with trimmed sugar chains (15 mg) was dissolved in 1 mL of 100 mM MES buffer (pH 6.5) containing 10 mm uridine-5′-diphospho-N-acetylglucosamine (UDP-GlcNAc), 20 mM manganese chloride, 10 mM sodium chloride, and 0.2 mM 2-mercaptoethanol, 1 mL of the immobilized β1,2-N-acetylglucosaminyltransferase I obtained in Example 14 was added, and a reaction was carried out for 24 hours as the contents were gently shaken at 25° C. After the reaction, the immobilized β1,2-N-acetylglucosaminyltransferase I was filtered off, and the immobilized β1,2-N-acetylglucosaminyl transferase I was washed with 5 mL of distilled water. The reaction solution and the wash were combined, and the mixture was introduced into a dialysis membrane (by Spectrum) with a 3.5 kD molecular weight cut off and was dialyzed against 50 mM HEPES buffer (pH 7.5) containing 10 mM manganese chloride. After the dialysis, it was concentrated to 1 mL using Ultrafree-MC (Millipore, molecular weight cut off 5,000).

Step 3: Elongation of Sugar Chains with Immobilized β1,4-Galactosyltransferase

UDP-Gal was added to a concentration of 10 mM in the solution obtained in step 2, 1 mL of the immobilized β1,4-galactosyltransferase obtained in Example 17 was then added, and a reaction was carried out for 24 hours as the contents were gently shaken at 25° C. After the reaction, a 1 mL solution of the target material was obtained in the same manner as in step 2.

Step 4: Elongation of Sugar Chains with Immobilized α2,6-Sialyltransferase

CMP-NeuAc was added to a concentration of 10 mM in 1 mL of the solution obtained in step 3, 1 mL of the immobilized α2,6-sialyltransferase obtained in Example 18 was then added, and a reaction was carried out for 48 hours as the contents were gently shaken at 25° C. The reaction was followed by dialysis against distilled water in the same manner as in step 2, and then by lyophilization, giving 10 mg of the target material.

(2) Analysis of Sugar Chain Conversion

Sugar chains were cut out in the usual manner from samples obtained in steps 1-4 above for fluorescent labeling with 2-aminopyridine. The resulting pyridylaminated oligosaccharides were analyzed by normal phase HPLC, confirming that the major component of the sugar chain structure in the lyophilized product obtained in step 4 was the structure represented by Formula 16.

### EXAMPLE 21

Use of Immobilized Enzyme for Conversion of RNaseB High Mannose-Type Sugar Chains to Complex-Type Sugar Chains

(1) Sugar Chain Conversion Reaction

Step 1: Trimming High Mannose-Type Sugar Chains With Immobilized α1,2-Mannosidase

RNaseB with trimmed sugar chains (87 mg) was obtained in the same manner as in step 1 in Example 20.

Step 2: Elongation of Sugar Chains with Immobilized 1,2-N-acetylglucosaminyltransferase I

The RNaseB with trimmed sugar chains (15 mg) was dissolved in 1 mL of 100 mM MES buffer (pH 6.5) containing UDP-GlcNAc (10 mM), 20 mM manganese chloride, 10 mM sodium chloride, and 0.2 mM 2-mercaptoethanol, the immobilized β1,2-N-acetylglucosaminyltransferase I (1 mL) obtained in Example 14 was added, and a reaction was carried out for 24 hours as the contents were gently shaken at 25° C. After the reaction, the immobilized β1,2-N-acetylglucosaminyltransferase I was filtered off, and the immobilized β1,2-N-acetylglucosaminyltransferase I was washed with 5 mL of distilled water. The reaction solution and the wash were combined, and the mixture was introduced into a dialysis membrane (by Spectrum) with a 3.5 kD molecular weight cut off and was dialyzed against 100 mM MES buffer (pH 6.0) containing 0.01 mM zinc acetate. After the dialysis, it was concentrated to 1 mL using Ultrafree-MC (Millipore, molecular weight cut off 5,000).

Step 3: Trimming Sugar Chains With Immobilized α-Mannosidase

1 mL of the immobilized α-mannosidase obtained in Example 19 was added to 1 mL of the reaction solution obtained in step 2, and a reaction was carried out for 24 hours as the contents were gently shaken at 25° C. After the reaction, a 1 mL solution of the target material was obtained in the same manner as in step 2 except dialysis against 100 mM MES buffer (pH 6.5) containing 20 mM manganese chloride, 10 mM sodium chloride, and 0.2 mM 2-mercaptoethanol.

Step 4: Elongation of Sugar Chains with Immobilized β1,2-N-acetylglucosaminyltransferase II

UDP-GlcNAc was added to a concentration of 10 mM in the solution obtained in step 3, 1 mL of the immobilized β1,2-N-acetylglucosaminyltransferase II obtained in Example 13 was added, and a reaction was carried out for 48 hours as the contents were gently shaken at 25° C. After the reaction, a 1 mL solution of the target material was obtained in the same manner as in step 2 except dialysis against 50 mM HEPES buffer (pH 7.5) containing 10 mM manganese chloride.

Step 5: Elongation of Sugar Chains with Immobilized β1,4-Galactosyltransferase

The sugar chains were elongated in the same manner as in step 3 in Example 20, giving a 1 mL solution of the target material.

Step 6: Elongation of Sugar Chains with Immobilized α2,6-Sialyltransferase

The sugar chains were elongated in the same manner as in step 4 in Example 20 except that the reaction time was 72 hours, giving 6 mg of the target material.

(2) Analysis of Sugar Chain Conversion

Sugar chains were cut out in the usual manner from samples obtained in steps 1-6 above for fluorescent labeling with 2-aminopyridine. The resulting pyridylaminated oligosaccharides were analyzed by normal phase HPLC, confirming that the major component of the sugar chain structure in the lyophilized product obtained in step 6 was the structure represented by Formula 17.

### EXAMPLE 22

Use of Immobilized Enzyme for Conversion of High Mannose-Type Sugar Chains to Complex-Type Sugar Chains

RNaseB sugar chains were converted in the same manner as in Example 21 except that the immobilized α-mannosidase II obtained in Example 16 was used instead of the immobilized α-mannosidase obtained in step 3 of Example 21. Analysis of the sugar chain structure of converted sugar chains which had been cut out confirmed that the major component was the structure represented by Formula 17.

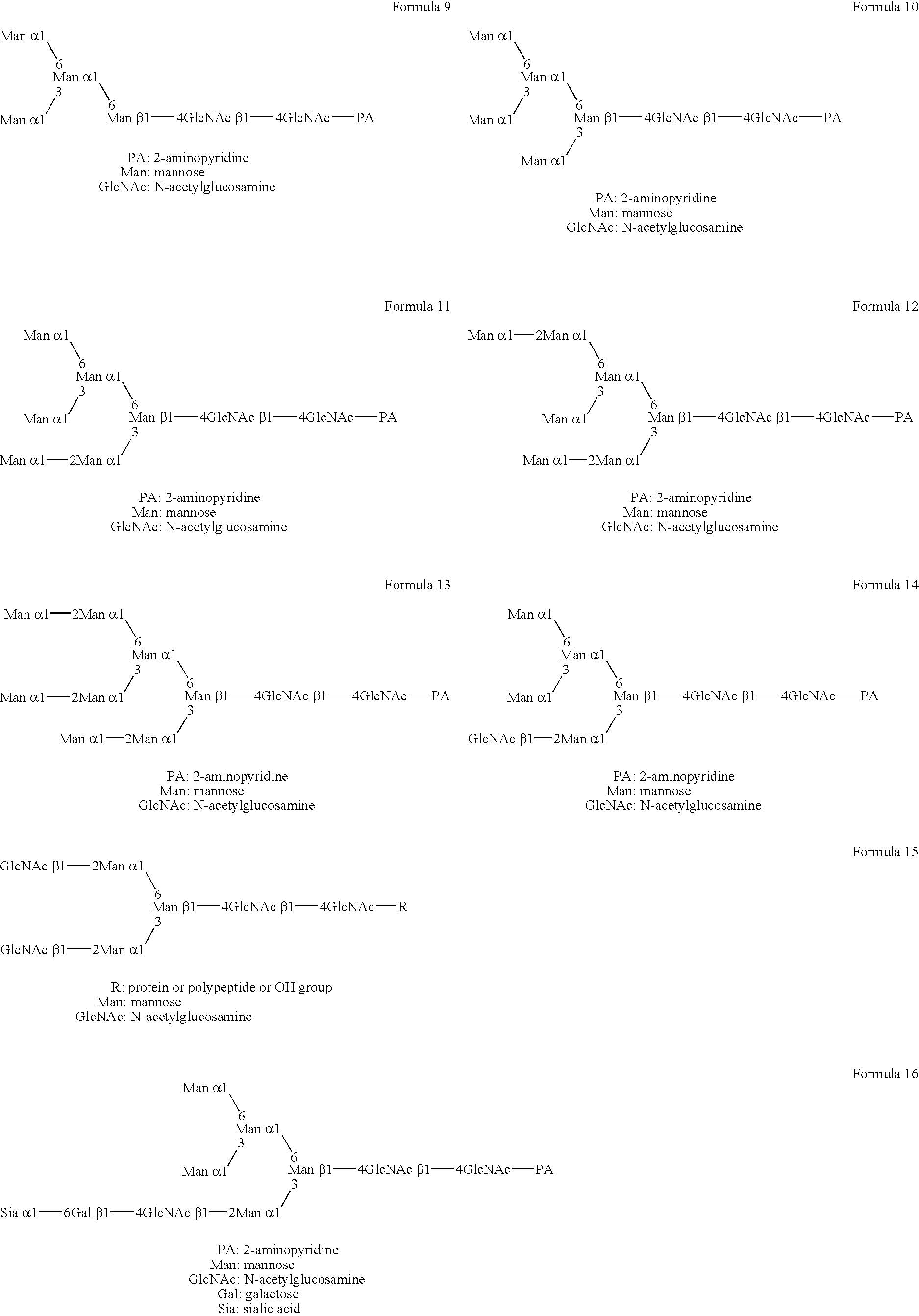

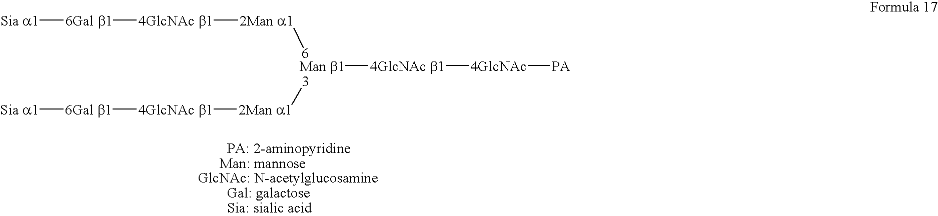

In the method of the present invention, a fusion protein having N-acetylglucosaminyltransferase II activity can be readily produced efficiently in large amounts in the form of a soluble protein. In addition, the N-acetylglucosaminyltransferase II itself can be readily obtained from the fusion protein, and the resulting N-acetylglucosaminyltransferase II allow to synthesize useful sugar cains and obtain anti N-acetylglucosaminyltransferase II antibody which can be useful for diagnostics, and the like.

1. A recombinant fusion protein of a sugar-binding protein and β1,2-N-acetylglucosaminyltransferase II.

2. A fusion protein according to claim 1, wherein the β1,2-N-acetylglucosaminyltransferase II is derived from humans.

3. A fusion protein according to claim 1, wherein the β1,2-N-acetylglucosaminyltransferase II is (a) a protein comprising the amino acid sequence in SEQ ID NO. 2, or (b) a protein with β1,2-N-acetylglucosaminyl transferase II activity, comprising the amino acid sequence in (a) above with one or more amino acids deleted, substituted, or added.

4. A protein according to claim 3, wherein the β1,2-N-acetylglucosaminyltransferase II comprises at least the amino acid sequence 20-447 in the amino acid sequence in SEQ ID NO. 2.

5. A fusion protein according to claim 1, wherein the β1,2-N-acetylglucosaminyltransferase II comprises an amino acid sequence in which amino acids corresponding to part or all of the protein transmembrane domain have been deleted.

6. A fusion protein according to claim 1, comprising a protease recognition site between the sugar-binding protein and the β1,2-N-acetylglucosaminyl transferase II.

7. A fusion protein according to claim 1, wherein the sugar-binding protein is a maltose-binding protein.

8. DNA coding for a fusion protein according to any of claims 1 through 7.

9. An expression vector comprising DNA according to claim 8.

10. A transformant resulting from transformation with an expression vector according to claim 9.

11. A method for producing a sugar-binding protein/β1,2-N-acetylglucosaminyltransferase II fusion protein, comprising the following steps of:
(1) transforming E. coli using an expression vector to which DNA coding for a sugar-binding protein and DNA coding for β1,2-N-acetylglucosaminyltransferase=II have been ligated in such a way that the two proteins are expressed in the form of a fusion protein under the control of a promoter capable of functioning in E. coli;
(2) cultivating the resulting transformants to produce a fusion protein of the sugar-binding protein and the β1,2-N-acetylglucosaminyltransferase II; and
(3) isolating the fusion protein from the resulting culture.

12. A method according to claim 11, wherein the β1,2-N-acetylglucosaminyltransferase II is derived from humans.

13. A method according to claim 12, wherein the DNA coding for the β1,2-N-acetylglucosaminyltransferase II comprises a nucleotide sequence coding for at least the amino acid sequence 29-447 in the amino acid sequence in SEQ ID NO. 2.

14. A method according to claim 12, wherein the DNA coding for the β1,2-N-acetylglucosaminyltransferase II comprises at least the nucleotide sequence 85-1341 in the nucleotide sequence in SEQ ID NO. 1.

15. A method according to claim 11, wherein the DNA coding for the β1,2-N-acetylglucosaminyltransferase II comprises a nucleotide sequence coding for an amino acid sequence in which amino acids corresponding to part or all of the protein transmembrane domain have been deleted.

16. A method according to claim 11, wherein the DNA coding for the β1,2-N-acetylglucosaminyltransferase II comprises the nucleotide sequence in SEQ ID NO. 1 from which amino acids corresponding to part or all of the protein transmembrane domain have been deleted.

17. A method according to claim 11, wherein the sugar-binding protein is a maltose-binding protein.

18. A method according to claim 17, wherein the DNA coding for the maltose-binding protein is derived from pMAL-p2 or pMAL-c2.

19. A method according to claim 11, wherein the fusion protein is isolated in the presence of divalent metal ions from the culture obtained in (3).

20. A method according to claim 19, wherein the divalent metal is manganese.

21. A method for producing β1,2-N-acetyl glucosaminyltransferase II, comprising the step of isolating the β1,2-N-acetylglucosaminyltransferase II after eliminating the sugar-binding protein portion from the fusion protein obtained by a method according to any of claims 11 through 20.

22. A method according to claim 21, characterized in that the DNA coding for the sugar-binding protein comprises a nucleotide sequence coding for a protease recognition site on the C terminal end of the protein, and the sugar-binding protein portion is eliminated from the fusion protein through the action of a protease.

23. A method according to claim 22, wherein the protease is blood coagulation factor Xa.

24. A method for converting sugar chains on glycoproteins to complex type sugar chains, comprising steps 1 through 4 below:
(step 1) allowing a glycosidase to act on glycoprotein sugar chains;
(step 2) allowing β1,2-N-acetylglucosaminyl transferase I to act, in the presence of UDP-GlcNAc, on the glycoproteins obtained in (step 1);
(step 3) allowing α-mannosidase to act on the glycoproteins obtained in (step 2); and
(step 4) allowing β1,2-N-acetylglucosaminyl transferase II to act, in the presence of UDP-GlcNAc, on the glycoproteins obtained in (step 3).

25. A method according to claim 24, characterized in that at least one kind of glycosyltransferase is furthermore allowed to act after step 4.

26. A method according to claim 25, wherein the glycosyltransferase is at least one selected from the group consisting of sialyltransferase, fucosyltransferase, galactosyltransferase, and N-acetylglucosaminyltransferase.

27. A method according to claim 25, wherein at least one glycosyltransferase is an immobilized enzyme.

28. A method according to claim 24, wherein the glycosidase is at least one selected from the group consisting of galactosidase, N-acetylglucosaminidase, fucosidase, sialidase, xylosidase, and mannosidase.

29. A method according to claim 24, wherein the glycosidase is α-mannosidase.

30. A method according to claim 24, wherein the glycosidase is α1,2-mannosidase.

31. A method according to claim 24, wherein the α-mannosidase is α-mannosidase II.

32. A method according to claim 24, wherein the β1,2-N-acetylglucosaminyltransferase II is a recombinant fusion protein according to any of claims 1 through 7, or a β1,2-N-acetylglucosaminyltransferase II from which the sugar-binding protein has been eliminated by cleavage at the protease recognition site.

33. A method according to claim 24, wherein at least one of the glycosidase, β1,2-N-acetyl glucosaminyltransferase I, α-mannosidase, or β1,2-N-acetylglucosaminyltransferase II is an immobilized enzyme.

34. A method according to claim 24, wherein the glycoprotein is naturally derived.

35. A method according to claim 24, wherein the glycoprotein is recombinant.

36. A method for converting sugar chains on glycoproteins to complex type sugar chains, comprising the following steps 1-3:
(step 1) allowing β1,2-N-acetylglucosaminyl transferase I to act, in the presence of UDP-GlcNAc, on glycoproteins having a structure wherein part or all of the sugar chain structures on the glycoproteins serve as the substrate for the β1,2-N-acetylglucosaminyl transferase I;
(step 2) allowing α-mannosidase to act on the glycoproteins obtained in (step 1); and
(step 3) allowing β1,2-N-acetylglucosaminyl transferase II to act, in the presence of UDP-GlcNAc, on the glycoproteins obtained in (step 2).

37. A method according to claim 36 for converting sugar chains on glycoproteins to complex type sugar chains, characterized in that at least one kind of glycosyltransferase is furthermore allowed to act after step 3.

38. A method according to claim 37, wherein the glycosyltransferase is at least one selected from the group consisting of sialyltransferase, fucosyltransferase, galactosyltransferase, and N-acetylglucosaminyltransferase.

39. A method according to claim 37, wherein at least one glycosyltransferase is an immobilized enzyme.

40. A method according to claim 36, wherein the α-mannosidase is α-mannosidase II.

41. A method according to claim 36, wherein the β1,2-N-acetylglucosaminyltransferase II is a recombinant fusion protein according to any of claims 1 through 7, or a β1,2-N-acetylglucosaminyltransferase II from which the sugar-binding protein has been eliminated by cleavage at the protease recognition site.

42. A method according to claim 36, wherein at least one of the β1,2-N-acetylglucosaminyltransferase I, α-mannosidase, or β1,2-N-acetylglucosaminyltransferase II is an immobilized enzyme.

43. A method according to claim 36, wherein the glycoprotein is naturally derived.

44. A method according to claim 36, wherein the glycoprotein is recombinant.

45. A method for converting sugar chains on glycoproteins to complex type sugar chains, comprising steps 1 through 3 below:
(step 1) allowing a glycosidase to act on glycoprotein sugar chains;
(step 2) allowing β1,2-N-acetylglucosaminyl transferase I to act, in the presence of UDP-GlcNAc, on the glycoproteins obtained in (step 1); and
(step 3) allowing β1,2-N-acetylglucosaminyl transferase II to act, in the presence of UDP-GlcNAc, on the glycoproteins obtained in (step 2).

46. A method according to claim 45, characterized in that at least one kind of glycosyltransferase is furthermore allowed to act after step 3.

47. A method according to claim 45, wherein the glycosyltransferase is at least one selected from the group consisting of sialyltransferase, fucosyltransferase, galactosyltransferase, and N-acetylglucosaminyltransferase.

48. A method according to claim 46, wherein at least one glycosyltransferase is an immobilized enzyme.

49. A method according to claim 45, wherein the glycosidase is at least one selected from the group consisting of galactosidase, N-acetylglucosaminidase, fucosidase, sialidase, xylosidase, and mannosidase.

50. A method according to claim 45, wherein the β1,2-N-acetylglucosaminyltransferase II is a recombinant fusion protein according to any of claims 1 through 7, or a β1,2-N-acetylglucosaminyltransferase II from which the sugar-binding protein has been eliminated by cleavage at the protease recognition site.

51. A method according to claim 45, wherein at least one of the glycosidase, β1,2-N-acetylglucosaminyltransferase I, or β1,2-N-acetylglucosaminyl transferase II is an immobilized enzyme.

52. A method according to claim 45, wherein the glycoprotein is naturally derived.

53. A method according to claim 45, wherein the glycoprotein is recombinant.

54. A method for converting hybrid-type sugar chains on glycoproteins to complex-type sugar chains, comprising steps 1 and 2 below:
(step 1) allowing a glycosidase to act on hybrid-type sugar chains of glycoproteins; and
(step 2) allowing β1,2-N-acetylglucosaminyl transferase II to act, in the presence of UDP-GlcNAc, on the glycoproteins obtained in (step 1).

55. A method according to claim 54, characterized in that at least one kind of glycosyltransferase is furthermore allowed to act after step 2.

56. A method according to claim 55, wherein the glycosyltransferase is at least one selected from the group consisting of sialyltransferase, fucosyltransferase, galactosyltransferase, and N-acetylglucosaminyltransferase.

57. A method according to claim 55, wherein at least one glycosyltransferase is an immobilized enzyme.

58. A method according to claim 54, wherein the glycosidase is at least one selected from the group consisting of mannosidase, xylosidase, fucosidase, and β1,4-N-acetylglucosaminidase.

59. A method according to claim 54, wherein the β1,2-N-acetylglucosaminyltransferase II is a recombinant fusion protein according to any of claims 1 through 7, or a β1,2-N-acetylglucosaminyltransferase II from which the sugar-binding protein has been eliminated by cleavage at the protease recognition site.

60. A method according to claim 54, wherein at least one of the glycosidase or β1,2-N-acetyl glucosaminyltransferase II is an immobilized enzyme.

61. A method according to claim 54, wherein the glycoprotein is naturally derived.

62. A method according to claim 54, wherein the glycoprotein is recombinant.

63. A method for converting high mannose-type sugar chains on glycoproteins into hybrid-type sugar chains, comprising the following steps 1 through 3, wherein at least one of the glycosidase, β1,2-N-acetylglucosaminyltransferase I, or β1,4-galactosyl transferase is an immobilized enzyme:
(step 1) allowing a glycosidase to act on high mannose-type sugar chains on glycoproteins;
(step 2) allowing β1,2-N-acetylglucosaminyl transferase I to act, in the presence of UDP-GlcNAc, on the glycoproteins obtained in (step 1); and
(step 3) allowing β1,4-galactosyltransferase to act, in the presence of UDP-Gal, on the glycoproteins obtained in (step 2).

64. A method according to claim 63 for converting sugar chains on glycoproteins to hybrid-type sugar chains, characterized in that at least one kind of glycosyltransferase is furthermore allowed to act after step 3.

65. A method according to claim 64, wherein the glycosyltransferase is at least one selected from the group consisting of sialyltransferase, fucosyltransferase, galactosyltransferase, xylosyltransferase, mannosyltransferase, and N-acetylglucosaminyltransferase.

66. A method according to claim 64, wherein at least one glycosyltransferase is an immobilized enzyme.

67. A method according to claim 63, wherein the glycosidase is at least one selected from the group consisting of galactosidase and α-mannosidase.

68. A method according to claim 63, wherein the α-mannosidase is α1,2-mannosidase.

69. A method according to claim 63, wherein the glycoprotein is naturally derived.

70. A method according to claim 63, wherein the glycoprotein is recombinant.


SEQUENCE LISTING (9 sequences)

SEQ ID NO: 1 - DNA, 1392 bp, Homo sapiens
  CDS (1)..(1341)
atgaggttccgcatctacaaacggaaggtgctaatcctgacgctcgtggtggccgcctgcggcttcgtcctctggagcagcaatgggcgacaaaggaagaacgaggccctcgccccaccgttgctggacgccgaacccgcgcggggtgccggcggccgcggtggggaccacccctctgtggctgtgggcatccgcagggtctccaacgtgtcggcggcttccctggtcccggcggtcccccagcccgaggcggacaacctgacgctgcggtaccggtccctggtgtaccagctgaactttgatcagaccctgaggaatgtagataaggctggcacctgggccccccgggagctggtgctggtggtccaggtgcataaccggcccgaatacctcagactgctgctggactcacttcgaaaagcccagggaattgacaacgtcctcgtcatctttagccatgacttctggtcgaccgagatcaatcagctgatcgccggggtgaatttctgtccggttctgcaggtgttctttcctttcagcattcagttgtaccctaacgagtttccaggtagtgaccctagagattgtcccagagacctgccgaagaatgccgctttgaaattggggtgcatcaatgctgagtatcccgactccttcggccattatagagaggccaaattctcccagaccaaacatcactggtggtggaagctgcattttgtgtgggaaagagtgaaaattcttcgagattatgctggccttatacttttcctagaagaggatcactacttagccccagacttttaccatgtcttcaaaaagatgtggaaactgaagcagcaagagtgccctgaatgtgatgttctctccctggggacctatagtgccagtcgcagtttctatggcatggctgacaaggtagatgtgaaaacttggaaatccacagagcacaatatgggtctagccttgacccggaatgcctatcagaagctgatcgagtgcacagacactttctgtacttatgatgattataactgggactggactcttcaatacttgactgtatcttgtcttccaaaattctggaaagtgctggttcctcaaattcctaggatctttcatgctggagactgtggtatgcatcacaagaaaacctgtagaccatccactcagagtgcccaaattgagtcactcttaaataataacaaacaatacatgtttccagaaactctaactatcagtgaaaagtttactgtggtagccatttccccacctagaaaaaatggagggtggggagatattagggaccatgaactctgtaaaagttatagaagactgcagtgaaaatcacagttacaaaagcgacagtcttctatttttgatatttgtcca

SEQ ID NO: 2 - protein, 447 aa, Homo sapiens
MRFRIYKRKVLILTLVVAACGFVLWSSNGRQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 3 - protein, 4 aa, Homo sapiens
IEGR

SEQ ID NO: 4 - DNA, 24 bp, Artificial
  Primer
aggttccgcatctacaaacggaag

SEQ ID NO: 5 - DNA, 27 bp, Artificial
  Primer
tgtcgcttttgtaactgtgattttcac

SEQ ID NO: 6 - DNA, 29 bp, Artificial
  Primer
aaggatccgggcgacaaaggaagaacgag

SEQ ID NO: 7 - DNA, 32 bp, Artificial
  Primer
aaaagcttgtaactgtgattttcactgcagtc

SEQ ID NO: 8 - DNA, 39 bp, Artificial
  Primer
tcgagctcgaacaacaacaacaataacaataacaacaac

SEQ ID NO: 9 - protein, 13 aa, Artificial
  Primer
SSSNNNNNNNNNN